(19) United States
(12) Patent Application Publication
(10) Pub. No.: US 2026/0167661 A1
(43) Pub. Date: Jun. 18, 2026

(54) RNA CAPPING EFFICIENCY ASSAY

(71) Applicant: BioNTech SE, Mainz (DE)

(72) Inventors: Maximilian Buff, Hofheim (DE); Christoph Kröner, Mainz (DE); Kevin Tritschler, Mainz (DE); Susanne Schweier, Mainz (DE); Anne Wurdak, Mainz (DE); Klaus Koch, Mainz (DE); Svenja Grobe, Mainz (DE)

(21) Appl. No.: 19/129,327

(22) PCT Filed: Nov. 10, 2023
(86) PCT No.: PCT/EP2023/081475

§ 371 Date: May 13, 2025

(30) Foreign Application Priority Data
Nov. 14, 2022 (WO) PCT/EP2022/081846

(51) Int. Cl. C07H 19/16

(52) U.S. Cl. CPC C07H 19/16

(57) ABSTRACT

A method of quantifying RNA capping efficiency, the method comprising: (a) providing a sample of capped RNA; (b) contacting the capped RNA with a nuclease, wherein the nuclease is a protein, thereby hydrolysing the RNA to produce hydrolysis products comprising a capped product comprising dinucleotides, and an uncapped product comprising nucleotides, step (b) being carried out in the absence of a nucleic acid having a base sequence complementary to the sequence of the capped RNA; (c) separating the hydrolysis products by chromatography; and (d) determining the concentrations of the hydrolysis products by triple quadrupole mass spectrometry, thereby quantifying RNA capping efficiency; is provided.

### TECHNICAL FIELD

This invention relates to a method of quantifying RNA capping efficiency.

### BACKGROUND TO THE INVENTION

Messenger RNA (“mRNA”) therapy is becoming an increasingly important approach for the treatment of a variety of diseases. Effective mRNA therapy requires effective delivery of the mRNA to the patient and efficient production of the protein encoded by the mRNA within the patient's body.

To optimize mRNA delivery and protein production in vivo, a proper cap is typically required at the 5′ end of the construct, which protects the mRNA from degradation and facilitates successful protein translation. The cap structure at the 5′ end of the mRNA can either be introduced co-transcriptionally with a so-called cap analogue, or with the help of an enzyme complex, e.g. Vaccinia Capping System (post-transcriptionally).

Accurate characterization of the proportion of capped RNA (capping efficiency) is particularly important for determining the quality of mRNA for therapeutic applications, to assess the expected translation efficiency and optimize the RNA production process.

WO2014/152659 and EP2971102B describe a capping assay in which the 5′ end of the mRNA is first hybridized using a DNA oligonucleotide probe complementary to a sequence in the 5′ untranslated region of the mRNA adjacent the cap such that the DNA anneals to the mRNA and treatment with one or more nucleases produces fragments of the 5′ end, which are then analyzed chromatographically such that the relative amounts of the capped and uncapped fragments are determined. However, this document does not disclose a method wherein the step of hydrolysing the capped RNA is carried out in the absence of a nucleic acid having a base sequence complementary to the sequence of the capped RNA. Moreover, this assay method suffers from the disadvantage that a complementary DNA probe first has to be produced and tested for its functionality for each different sequence of the 5′ end. The need to synthesise a complementary DNA for every capped RNA sequence to be analysed makes the method slow, inefficient and difficult to adapt to different capped mRNAs. Moreover, since the transcribed RNA is not homogeneous at the 5′ end, the peaks found in the chromatogram must be assigned to capped or uncapped species.

Beverly et al. Anal. Bioanal. Chem., 2016, 408 (18), 5021-30 describes a similar method which comprises the hybridization of the 5′ end of the RNA with an RNA oligonucleotide that contains a few DNA nucleotides at the 3′ end. This creates a short (approximately 6 nucleotides) DNA-RNA hybrid double strand that can be specifically hydrolysed in this part using an RNaseH enzyme. This hydrolysis results in a 5′ fragment of the same length, which can be isolated using a biotin tag on the probe and examined using chromatography coupled to mass spectrometry. However, this method suffers from the same disadvantages as that of WO2014/152659 in that, for each different sequence at the 5′ end of the mRNA, a complementary RNA probe first has to be produced and tested for its functionality, and the hydrolysis fragment is not homogeneous.

Moreover, in the methods described in both documents, the evaluation is disturbed by transcripts that are one nucleotide shorter or longer. Depending on which cap analogue is used, these can arise: in particular, with a start sequence of two or three guanosines, as is recommended for efficient transcription. In addition, short truncation transcripts, which arise due to the natural mechanism of T7 RNA polymerase, can affect the assay. Furthermore, a new cleavage oligonucleotide must be designed and verified for each new 5′ sequence in both methods. This could be problematic for very structured sequences.

Trotman et al. Bio Protoc. 2018, 8 (6), e2767) describes enzymatic capping with radioactively labelled guanosine triphosphate (GTP). In order to determine the efficiency of the subsequent enzymatic methylation, the RNA is first hydrolysed with nuclease P1 and the degradation products are separated using thin-layer chromatography. Quantification of the methylated and non-methylated Cap species is based on the radioactivity. However, only the efficiency of the methyltransferase is determined and the method requires the use of radioactive labelled products, meaning that additional safety protocols need to be followed when carrying out the method.

WO2017/149139 describes a method for analyzing a sample comprising mRNA molecules which includes completely hydrolyzing the RNA molecules, thereby releasing nucleosides; and then separating and quantifying the released nucleosides by HPLC. In order to completely hydrolyse the mRNA, a phosphodiesterase I from Crotalus adamanteus and a shrimp alkaline phosphatase is added to a nuclease P1. However, this method requires the use of three separate enzymes, thereby requiring a number of further enzymatic steps and increasing the risk of cross-reactivity between the enzymes. Furthermore, the method would require the determination of the number of copies of the RNA in the sample to make the % capping calculation: this additional calculation step introduces a further level of uncertainty.

Muthmann et al. Methods, 2022, 203, 196-206, describes a method for quantifying mRNA cap modifications. The method described includes completely hydrolyzing the RNA molecules into nucleosides, using first a nuclease P1 together with a snake venom phosphodiesterase, followed by dephosphorylation with an alkaline phosphatase. The cap modifications are then quantified by liquid chromatography coupled to triple quadrupole mass spectrometry. However, this method also requires the use of three separate enzymes, thereby requiring a number of further enzymatic steps and increasing the risk of cross-reactivity between the enzymes. Furthermore, this method is aimed at determining the modification levels of the mRNA, rather than the capping efficiency, and consequently requires the quantification of different analytes.

US2020/0032274 discloses synthetic thermostable polynucleotides, and also describes in general terms that capped polynucleotides may be treated with nuclease to yield a mixture of free nucleotides and the capped 5′,5-triphosphate cap structure be detected by LC-MS—the amount of capped product on the LC-MS spectra corresponding to capping reaction efficiency. This document does not disclose such a method wherein the concentrations of the hydrolysis products are determined by triple quadrupole mass spectrometry.

EP3090060B describes a method to measure RNA capping efficiency by hydrolysing RNA with hammerhead ribozyme HHNUH2d, which is an RNA motif that catalyses reversible cleavage and ligation reactions at a specific site within an RNA molecule. The products may be separated by HPLC, and the presence or absence of a cap structure may be determined using a number of methods, including quantitative mass spectrometry. This document does not disclose such a method wherein the agent used to hydrolyse the RNA is a protein. Moreover, the methods described in this document suffer from the drawback that a specific ribozyme or probe would have to be designed for each RNA sequence to be analysed.

It would be desirable to provide a simplified method for determining the capping efficiency of RNA, which is applicable to RNA having any sequence of the 5′ end, without the need to provide a complementary nucleic acid probe specific to every RNA to be analysed, and which can be a clear, standardized evaluation, and no radioactive labelling is required. It would also be desirable for this to be carried out using a single enzyme rather than multiple enzymes to hydrolyse the RNA.

These objectives are fulfilled by the present invention as defined and described herein.

### SUMMARY OF THE INVENTION

According to one aspect of the invention, there is provided a method of quantifying RNA capping efficiency, the method comprising:
(a) providing a sample of capped RNA;
(b) contacting the capped RNA with a nuclease, thereby hydrolysing the RNA to produce hydrolysis products comprising a capped product comprising dinucleotides, and an uncapped product comprising nucleotides;
(c) separating the hydrolysis products by chromatography; and
(d) determining the concentrations of the hydrolysis products by mass spectrometry, thereby quantifying RNA capping efficiency.

According to another aspect of the invention, there is provided a method of quantifying RNA capping efficiency, the method comprising the following steps (a) to (d):
(a) providing a sample of capped RNA;
(b) contacting the capped RNA with a nuclease, wherein the nuclease is a protein, thereby hydrolysing the RNA to produce hydrolysis products comprising a capped product comprising dinucleotides, and an uncapped product comprising nucleotides, step (b) being carried out in the absence of a nucleic acid having a base sequence complementary to the sequence of the capped RNA;
(c) separating the hydrolysis products by chromatography; and
(d) determining the concentrations of the hydrolysis products by triple quadrupole mass spectrometry, thereby quantifying RNA capping efficiency.

### Advantages and Surprising Findings

It has surprisingly been found by the present inventors that a capped RNA assay comprising hydrolysis of the capped RNA using a nuclease as the sole hydrolysing enzyme, thereby producing a hydrolysis product comprising nucleotides, followed by determination of the analytes by chromatography (especially liquid chromatography) coupled to mass spectrometry (especially tandem mass spectrometry) enables the capped and uncapped products to be determined, and therefore the RNA capping efficiency to be determined, in a manner which is simple and applicable to any RNA sequence, and can be achieved in a highly selective, flexible and sensitive manner. The method can be used for a wide variety of different RNA cap structures to determine the RNA capping efficiency. There is no need to first prepare a nucleic acid probe complementary to the RNA as the hybridisation step is eliminated. The assay method only requires a corresponding external standard to be produced.

In particular, it has been found by the present inventors that determination of the analytes using hydrophilic interaction chromatography (HILIC) in combination with mass spectroscopy using a triple quadrupole mass spectrometer is particularly advantageous for the quantitation of nucleotides. HILIC makes it possible to retain and separate very polar small analytes such as nucleotides. The triple quadrupole mass spectrometer, particularly although not exclusively when acting in multiple reaction monitoring (MRM) mode, enables highly sensitive, selective, accurate and universal detection of nucleotides, compared with non-selective detection, for example using ultraviolet spectroscopy. The combination of HILIC and tandem mass spectroscopy (especially when acting in MRM mode) enables a very selective, sensitive, and accurate measurement of nucleotides in complex matrices.

Furthermore, in contrast to the methods described in WO2017/149139 and Muthmann et al. as described above, the method can be carried out using a single enzyme rather than multiple enzymes, thereby simplifying the method by reducing the number of enzymatic steps and lessening or eliminating the risk of cross-reactivity between the enzymes. In particular, the method of the present invention exhibits the advantage, compared with those described in Muthmann et al., in that it directly measures the nucleotides resulting from hydrolysis of the RNA with the nuclease, and therefore avoids the need for use of an alkaline phosphatase to hydrolyse the nucleotides into nucleosides.

In addition, in contrast to the methods described in EP3090060B, which use a hammerhead ribozyme to hydrolyse the RNA, the use of a nuclease which is a protein according to the present invention means the method is equally applicable to any RNA sequence, thereby avoiding the need to design a specific ribozyme or probe for each RNA sequence.

### BRIEF DESCRIPTION OF THE FIGURES

FIG. 1 is an LC chromatogram showing the products of RNA capped with β-S-ARCA hydrolysed using the method of Example 1 and analysed using the method of Example 2;

FIG. 2 is an LC chromatogram showing the products of RNA capped with CleanCap® 413, hydrolysed using the method of Example 1 and analysed using the method of Example 2;

FIG. 3 is an LC chromatogram of the standard nucleotides and dinucleotides used for comparison with FIGS. 1 and 2; and

FIG. 4 shows the capping efficiency of varying cap amounts in in vitro transcription; and

FIG. 5 shows the capping efficiency of RNAs having two different cap structures, wherein “CC413 Cap” means CleanCap® 413 as defined herein, and “DI Cap” means β-S-ARCA.

### DETAILED DESCRIPTION

### Definitions

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims.

Unless defined otherwise, all technical and scientific terms used herein have the same meanings as commonly understood by one of ordinary skill in the art. Preferably, the terms used herein are defined as described in “A multilingual glossary of biotechnological terms: (IUPAC Recommendations)”, H. G. W. Leuenberger, B. Nagel, and H. Kolbl, Eds., Helvetica Chimica Acta, Basel, Switzerland, (1995). The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein.

The term “absent”, when used in relation to an enzyme composition, preferably means that the stated enzyme is not present in the enzyme composition. However, it is to be understood that trace amounts of the enzyme stated to be absent from the composition (such as less than 1%, less than 0.5%, less than 0.1%, less than 0.05%, less than 0.01%, less than 0.05%, less than 0.01%, less than 0.005%, by weight of the total weight of the enzyme composition) may be present in the enzyme composition provided that they do not affect the course of the enzymatic reaction or give rise to undesired side reactions. In one embodiment, the term “absent” means that the substance is not added to the composition.

“Alkyl” refers to straight chain and branched saturated hydrocarbon groups, generally having a specified number of carbon atoms (e.g., C₁₋₄ alkyl refers to an alkyl group having 1 to 4 (i.e., 1, 2, 3 or 4) carbon atoms, C₁₋₆ alkyl refers to an alkyl group having 1 to 6 carbon atoms, and so on). Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like. In some embodiments, alkyl means C₁₋₆ alkyl. In some embodiments, alkyl means C₁₋₄ alkyl. In some embodiments, alkyl means C₁₋₃ alkyl. In some embodiments, alkyl means C₁₋₂ alkyl.

“Halo,” “halogen” and “halogeno” may be used interchangeably and refer to fluoro, chloro, bromo, and iodo.

Anneal or hybridization: As used herein, the terms “anneal,” “hybridization,” and grammatical equivalent, refers to the formation of complexes (also called duplexes or hybrids) between nucleotide sequences which are sufficiently complementary to form complexes via Watson-Crick base pairing or non-canonical base pairing. It will be appreciated that annealing or hybridizing sequences need not have perfect complementary to provide stable hybrids. In many situations, stable hybrids will form where fewer than about 10% of the bases are mismatches. Accordingly, as used herein, the term “complementary” refers to a nucleic acid molecule that forms a stable duplex with its complement under particular conditions, generally where there is about 90% or greater homology (e.g., about 95% or greater, about 98% or greater, or about 99% or greater homology). Those skilled in the art understand how to estimate and adjust the stringency of hybridization conditions such that sequences that have at least a desired level of complementarity will stably hybridize, while those having lower complementarity will not. For examples of hybridization conditions and parameters, see, for example, Sambrook et al, “Molecular Cloning: A Laboratory Manual”, 1989, Second Edition, Cold Spring Harbor Press: Plainview, NY and Ausubel, “Current Protocols in Molecular Biology”, 1994, John Wiley & Sons: Secaucus, NJ. Complementarity between two nucleic acid molecules is said to be “complete”, “total” or “perfect” if all the nucleic acid's bases are matched, and is said to be “partial” otherwise.

Chromatography: As used herein, the term “chromatography” generally refers to a technique for separation of mixtures. Typically, the mixture is dissolved in a fluid called the “mobile phase,” or eluent, which carries it through a structure holding another material called the “stationary phase.” More specific chromatography techniques are defined in more detail herein.

Nucleoside: The term “nucleoside”, as used herein, refers to a nucleobase, which may be adenine (“A”), guanine (“G”), cytosine (“C”), uracil (“U”), thymine (“T”) linked to a carbohydrate, for example D-ribose (in RNA—the unit being termed a “ribonucleoside”) or 2′-deoxy-D-ribose (in DNA—the unit being termed a “deoxyribonucleoside”), through a glycosidic bond between the anomeric carbon of the carbohydrate (1′-carbon atom of the carbohydrate) and the nucleobase. When the nucleobase is purine, e.g., A or G, the ribose sugar is generally attached to the N9-position of the heterocyclic ring of the purine. When the nucleobase is pyrimidine, e.g., C, T or U, the sugar is generally attached to the N1-position of the heterocyclic ring. The carbohydrate may be substituted or unsubstituted. Substituted ribose sugars include, but are not limited to, those in which one or more of the carbon atoms, for example the 2′-carbon atom, is substituted with one or more of the same or different Cl, F, R, OR, NR₂ or halogen groups, where each R is independently H, C₁-C₆ alkyl or C₅-C₁₄ aryl. Ribose examples include ribose, 2′-deoxyribose, 2′,3′-dideoxy-ribose, 2′-haloribose, 2′-fluororibose, 2′-chlororibose, and 2′-alkylribose, e.g., 2′-O-methyl, 4′-alpha-anomeric nucleotides, 1′-alpha-anomeric nucleotides (Asseline et al, Nucl. Acids Res., 1991, 19, 4067-74) 2′-O-[2-(N-methylcarbamoyl)ethyl]ribose (Yamada et al., J. Org. Chem. 2011, 76, 3042-53).

Nucleoside Analogue: The term “nucleoside analogue”, as used herein, is intended to encompass compounds in which the carbohydrate portion of the nucleoside is replaced with a non-natural group. In one embodiment, the 2′-O and 4′-C or the 3′O- and 4′C positions of the ribose group are linked by a covalent bond or linker (typically a methylene or ethylene group)—such groups are termed “locked nucleic acids” or “LNA” The structures of LNAs can be found, for example, in Wengel, et al., Chemical Communications (1998) 455; Koshkin et al., Tetrahedron (1998) 54:3607; Jesper Wengel, Accounts of Chem. Research (1999) 32:301; Obika, et al., Tetrahedron Letters (1997) 38:8735; Obika, et al., Tetrahedron Letters (1998) 39:5401; and Obika, et al., Bioorganic Medicinal Chemistry (2008) 16:9230, and in WO 98/22489; WO 98/39352 and WO 99/14226).

In other embodiments, the carbohydrate moiety of the nucleotide is replaced with an N-(2-aminoethyl)glycine unit-such groups are termed “peptide nucleic acids” or “PNA”. PNAs can be produced synthetically using any technique known in the art. See, e.g., U.S. Pat. Nos. 6,969,766; 7,211,668; 7,022,851; 7,125,994; 7,145,006; and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991.

In other embodiments, the C2′-C3′ bond of the carbohydrate moiety has been cleaved-such groups are termed “unlocked nucleic acid” or “UNA” moieties. UNAs are disclosed, for example, in WO 2016/070166.

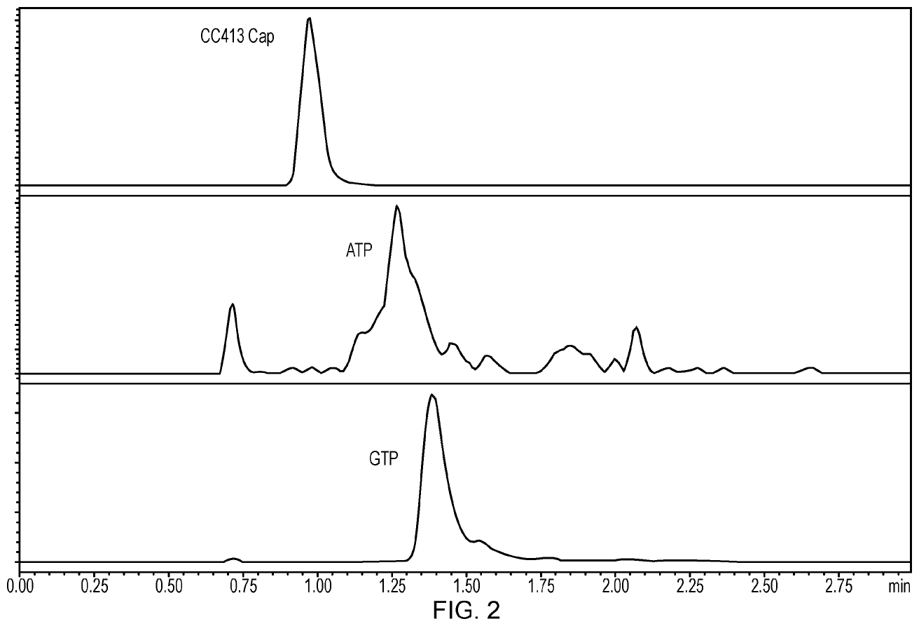

In other embodiments, the carbohydrate moiety of the nucleotide is replaced with a morpholino group, the nucleobase being present at the 3-position of the morpholino group and the 6-position of the adjacent morpholino group linked (via a —CH₂—O-linkage) to the phosphorus of the intersubunit linkage, which is in turn linked to the nitrogen of the adjacent morpholino group. Typically, in such compounds, the negatively charged oxygen of the phosphate intersubunit linkage is replaced by an amide or substituted amide group-such compounds having both the morpholino backbone and phosphorodiamidate inter-subunit linkage are termed “phosphorodiamidate morpholino” (or simply “morpholino” groups). Their general structure is as described in FIG. 2 of Summerton, J., et al., Antisense & Nucleic Acid Drug Development, 7:187-195 (1997) and their synthesis, structures, and binding characteristics of morpholino oligomers are detailed in U.S. Pat. Nos. 5,698,685; 5,217,866; 5,142,047; 5,034,506; 5,166,315; 5,521,063; 5,506,337; 8,076,476; and 8,299,206.

Nucleotide: The term “nucleotide” as used herein means a nucleoside (or nucleoside analogue) in a phosphorylated form (a phosphate ester of a nucleoside or nucleoside analogue), as a monomer unit or within a polynucleotide polymer. The phosphate group may be present at any oxygen on the sugar portion of the nucleotide. Typically, the phosphate group is present on the 3′-position or the 5′-position, preferably the 5′-position. The phosphate group may comprise any number of phosphate units, typically 1, 2, 3, 4, 5, 6, 7, 8, 9, or 10 phosphate units. Preferably, the phosphate group is a monophosphate (1 phosphate unit), diphosphate (2 phosphate units) or triphosphate (3 phosphate units). Sulfur may substitute for oxygen in any or all of the phosphate groups to form a thiophosphate group. “Nucleotide 5′-triphosphate” refers to a nucleotide with a triphosphate ester group at the 5′ position, sometimes denoted as “NTP”, or “dNTP” and “ddNTP” to particularly point out the structural features of the ribose sugar. The triphosphate ester group may include sulfur substitutions for the various oxygen moieties, e.g., alpha-thio-nucleotide 5′-triphosphates. Nucleotides can exist in the mono-, di-, or tri-phosphorylated forms. The carbon atoms of the ribose present in nucleotides are designated with a prime character (′) to distinguish them from the backbone numbering in the bases. For a review of polynucleotide and nucleic acid chemistry see Shabarova, Z. and Bogdanov, A. Advanced Organic Chemistry of Nucleic Acids, VCH, New York, 1994.

Dinucleotide: The term “dinucleotide” as used herein means a nucleic acid comprising two nucleosides, or nucleoside analogues (as defined above) connected by a mono- or poly-phosphate ester group. The phosphate group may be present at any oxygen on the sugar portion of the nucleotide. The phosphate group may comprise any number of phosphate units, typically 1, 2, 3, 4, 5, 6, 7, 8, 9, or 10 phosphate units. Preferably, the phosphate group is a monophosphate (1 phosphate unit), diphosphate (2 phosphate units), triphosphate (3 phosphate units) or tetraphosphate (4 phosphate units). Any one or all of the oxo (═O) groups in the phosphate units (preferably only 1 oxo group in 1 phosphate unit) may be replaced with a thio (═S) group. The phosphate group may independently be present on the 3′-position or the 5′-position of each nucleoside, and is preferably present at the 5′-position of both nucleosides. In one embodiment, the dinucleotide comprises two nucleosides connected by a 5′,5′-triphosphate bridge.

Nucleic acid: The terms “nucleic acid”, “nucleic acid molecule”, “polynucleotide” or “oligonucleotide” may be used herein interchangeably. They refer to polymers of nucleotide monomers or analogues thereof, such as deoxyribonucleic acid (DNA) and ribonucleic acid (RNA) and combinations thereof. The nucleotides may be genomic, synthetic or semi-synthetic in origin, and may contain nucleosides (as defined above) or nucleoside analogues (as defined above). encompassing nucleic acid-like structures with synthetic backbones, as well as amplification products. As will be appreciated by one skilled in the art, the length of these polymers (i.e., the number of nucleotides it contains) can vary widely, often depending on their intended function or use. Polynucleotides can be linear, branched linear, or circular molecules. Polynucleotides also have associated counter ions, such as H⁺, NH₄⁺, trialkylammonium, Mg²⁺, Na⁺ and the like. A polynucleotide may be composed entirely of deoxyribonucleotides, entirely of ribonucleotides, or chimeric mixtures thereof, or of nucleotides containing nucleoside analogues. Polynucleotides may be composed of inter-nucleotide nucleobase and sugar analogues.

In some embodiments, the term “oligonucleotide” is used herein to denote a polynucleotide that comprises between about 5 and about 150 nucleotides, e.g., between about 10 and about 100 nucleotides, between about 15 and about 75 nucleotides, or between about 15 and about 50 nucleotides.

Throughout the specification, whenever an oligonucleotide is represented by a sequence of letters (chosen, for example, from the four base letters: A, C, G, and T, which denote adenosine, cytidine, guanosine, and thymidine, respectively), the nucleotides are presented in the 5′ to 3′ order from the left to the right. A “polynucleotide sequence” refers to the sequence of nucleotide monomers along the polymer. Unless denoted otherwise, whenever a polynucleotide sequence is represented, it will be understood that the nucleotides are in 5′ to 3′ orientation from left to right.

Nucleic acids, polynucleotides and oligonucleotides may be comprised of standard nucleotide bases or substituted with nucleotide isoform analogues, including, but not limited to iso-C and iso-G bases, which may hybridize more or less permissibly than standard bases, and which will preferentially hybridize with complementary isoform analogue bases. Many such isoform bases are described, for example, by Benner et al, Cold Spring Harb. Symp. Quant. Biol. 1987, 52, 53-63. Analogues of naturally occurring nucleotide monomers include, for example, 7-deazaadenine, 7-deazaguanine, 7-deaza-8-azaguanine, 7-deaza-8-azaadenine, 7-methyl-guanine, inosine, nebularine, nitropyrrole (Bergstrom, J. Amer. Chem. Soc, 1995, 117, 1201-1209), nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine (Seela, U.S. Pat. No. 6,147,199), 7-deazaguanine (Seela, U.S. Pat. No. 5,990,303), 2-azapurine (Seela, WO 01/16149), 2-thiopyrimidine, 6-thioguanine, 4-thiothymine, 4-thiouracil, O-6-methylguanine, N-6-methyladenine, O-4-methylthymine, 5,6-dihydro-thymine, 5,6-dihydrouracil, 4-methylindole, pyrazolo[3,4-d]pyrimidines, “PPG” (Meyer, U.S. Pat. Nos. 6,143,877 and 6,127,121; Gall, WO 01/38584), and ethenoadenine (Fasman (1989) in Practical Handbook of Biochemistry and Molecular Biology, pp. 385-394, CRC Press, Boca Raton, Fla.).

The term “3”′ refers to a region or position in a polynucleotide or oligonucleotide 3′ (i.e., downstream) from another region or position in the same polynucleotide or oligonucleotide. The term “5”′ refers to a region or position in a polynucleotide or oligonucleotide 5′ (i.e., upstream) from another region or position in the same polynucleotide or oligonucleotide. The terms “3′ end” and “3′ terminus”, as used herein in reference to a nucleic acid molecule, refer to the end of the nucleic acid which contains a free hydroxyl group attached to the 3′ carbon of the terminal pentose sugar. The term “5′ end” and “5′ terminus”, as used herein in reference to a nucleic acid molecule, refers to the end of the nucleic acid molecule which contains a free hydroxyl or phosphate group attached to the 5′ carbon of the terminal pentose sugar. In some embodiments of the invention, oligonucleotide primers comprise tracts of poly-adenosine at their 5′ termini.

Step (a)—Provision of Capped RNA

The method of the present invention begins with the provision of a capped RNA. The capped RNA may be any capped RNA, either natural or synthetic. Typically, the RNA comprises nucleotides in which a ribose sugar has a base attached to the 1′ position, and a phosphate group which may be attached at the 5′-position or the 3′-position. The base may be adenine (A), cytosine (C), guanine (G) or uracil (U). Typically, the RNA is capped mRNA.

The cap may have any structure, natural or synthetic, which is capable of performing the function of binding to the cap-binding complex and EIF4E enabling the RNA to undergo translation during protein synthesis and/or protecting the RNA from degradation via 5′-3′ exonucleases.

In one embodiment, the cap has a structure of formula (I):

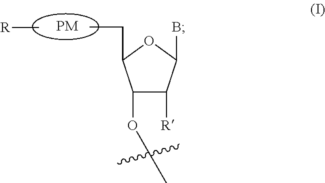

wherein:
PM is a mono- or polyphosphate moiety containing 1, 2, 3, 4, 5, 6, 7, 8, 9 or 10 phosphate units, the oxo (═O) groups in any of the phosphate units being optionally replaced with a thio (═S) group;
R is an end-cap moiety;
B is a nucleobase, optionally alkylated on a nitrogen atom by a C₁₋₄ alkyl group;
R′ is selected from OH, O(C₁₋₄ alkyl), and halogen; and
the squiggly line represents the rest of the RNA molecule.

In formula (I), R may represent any group capable of allowing the cap to perform the above-mentioned function of binding to the cap-binding complex and EIF4E enabling the RNA to undergo translation during protein synthesis and/or protecting the RNA from degradation via 5′-3′ exonucleases.

In one embodiment, the cap has a structure of formula (Ia):

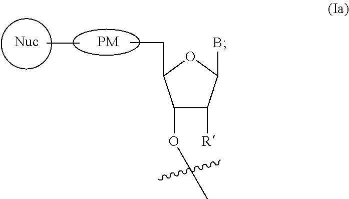

wherein:
PM is a mono- or polyphosphate moiety containing 1, 2, 3, 4, 5, 6, 7, 8, 9 or 10 phosphate units, the oxo (═O) groups in any of the phosphate units being optionally replaced with a thio (═S) group;
Nuc is a nucleoside or nucleoside analogue;
B is a nucleobase, optionally alkylated on a nitrogen atom by a C₁₋₄ alkyl group;
R′ is selected from OH, O(C₁₋₄ alkyl), and halogen; and
the squiggly line represents the rest of the RNA molecule.

In formulae (I) and (Ia), PM is preferably a monophosphate (1 phosphate unit), diphosphate (2 phosphate units), triphosphate (3 phosphate units) or tetraphosphate (4 phosphate units). Any one or all of the oxo (═O) groups in the phosphate units (preferably only 1 oxo group in 1 phosphate unit) may be replaced with a thio (═S) group.

In one embodiment of formula (Ia), Nuc is a nucleoside, which may be a ribonucleoside or deoxyribonucleoside (as defined above).

In another embodiment of formula (Ia), Nuc is a nucleoside analogue, as defined above. The nucleoside analogue may comprise a locked nucleic acid (LNA) moiety, a peptide nucleic acid (PNA) moiety, an unlocked nucleic acid (UNA) moiety or a morpholino moiety, as defined above.

In one embodiment of either formula (I) or (Ia), R′ is OH or OCH₃.

In one embodiment, the cap has a structure of formula (Ib):

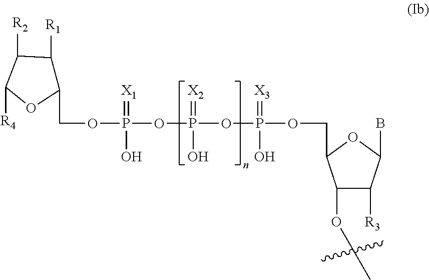

or a salt thereof, wherein:
B is a nucleobase, optionally alkylated on a nitrogen atom by a C₁₋₄ alkyl group;
R₁ is selected from OH, O(C₁₋₄ alkyl), and halogen;
R₂ is selected from H, OH, and O(C₁₋₄ alkyl), and halogen;
R₃ is selected from OH, O(C₁₋₄ alkyl), and halogen;
R₄ is H, OH, O(C₁₋₄ alkyl), halogen, or a nucleobase, optionally alkylated on a nitrogen atom by a C₁₋₄ alkyl group;
n is 1, 2 or 3;
X₁, each X₂, and X₃, are each independently O or S; and
the squiggly line represents the rest of the RNA molecule.

In one embodiment of formula (Ib), R₄ is OH. In one embodiment of formula (Ib), R₄ is a nucleobase, optionally alkylated on a nitrogen atom by a C₁₋₄ alkyl group.

In one embodiment, the cap has a structure of formula (Ic):

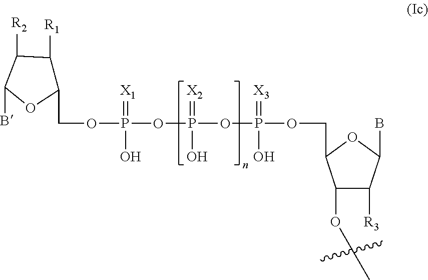

or a salt thereof, wherein:
B and B′ are each independently nucleobases, each optionally alkylated on a nitrogen atom by a C₁₋₄ alkyl group;
R₁ is selected from OH, O(C₁₋₄ alkyl), and halogen;
R₂ is selected from H, OH, and O(C₁₋₄ alkyl), and halogen;
R₃ is selected from OH, O(C₁₋₄ alkyl), and halogen;
n is 1, 2 or 3;
X₁, each X₂, and X₃, are each independently O or S; and the squiggly line represents the rest of the RNA molecule.

In one embodiment of formula (Ic), B is selected from adenine (“A”), guanine (“G”), cytosine (“C”), or uracil (“U”), each optionally alkylated on a nitrogen atom by a C₁₋₄ alkyl group, such as by a methyl group. In one embodiment of formula (Ic), B is G, optionally methylated on the nitrogen at the 7′-position.

In one embodiment of either formula (Ib) or (Ic), B′ is selected from adenine (“A”), guanine (“G”), cytosine (“C”), or uracil (“U”), each optionally alkylated on a nitrogen atom by a C₁₋₄ alkyl group, such as by a methyl group. In one embodiment of either formula (Ib) or (Ic), B′ is G, optionally methylated on the nitrogen at the 7′-position.

In one embodiment of either formula (Ib) or (Ic), R₁ is OH or OCH₃.

In one embodiment of either formula (Ib) or (Ic), R₂ is H, OH, or OCH₃.

In one embodiment of either formula (Ib) or (Ic), R₃ is OH or OCH₃.

In one embodiment of either formula (Ib) or (Ic), n is 1.

In one embodiment of either formula (Ib) or (Ic), X₁ is O. In one embodiment of either formula (Ib) or (Ic), X₃ is O. In one embodiment of either formula (Ib) or (Ic), X₁ is S. In one embodiment of either formula (Ib) or (Ic), X₃ is S. In one embodiment of either formula (Ib) or (Ic), each X₂ is O. In one embodiment of either formula (Ib) or (Ic), each X₂ is S.

In one embodiment of formula (Ic), B and B′ are both G, each optionally methylated on the nitrogen at the 7′-position. In one embodiment of formula (Ic), B is G, and B′ is 7′-methyl-G.

In one embodiment, of either formula (Ib) or (Ic), n is 1, X₁ and X₃ are O, and X₂ is O. In one embodiment of either formula (Ib) or (Ic), n is 1, X₁ and X₃ are O, and X₂ is S.

In one embodiment, the cap is a naturally occurring cap structure. One example of a naturally occurring cap structure is a 7-methyl guanosine that is linked via a triphosphate bridge to the 5′-end of the first transcribed nucleotide, resulting in a dinucleotide cap of m⁷ G (5′)ppp(5′)N, where Nis any nucleoside. This cap is a structure of formula (Ic) in which B′ is 7-methyl-G; n is 1, each X is O, X′ is O; and R₁, R₂ and R₃ are all OH.

In vivo, the cap is added enzymatically. The cap is added in the nucleus and is catalyzed by the enzyme guanylyl transferase. The addition of the cap to the 5′ terminal end of RNA occurs immediately after initiation of transcription. The terminal nucleoside is typically a guanosine, and is in the reverse orientation to all the other nucleotides, i.e., G(5′)ppp(5′)GpNpNp.

A common cap for mRNA produced by in vitro transcription is m⁷G(5′)ppp(5′) G, which has been used as the dinucleotide cap in transcription with T7 or SP6 RNA polymerase in vitro to obtain RNAs having a cap structure in their 5′-termini. The prevailing method for the in vitro synthesis of capped mRNA employs a pre-formed dinucleotide of the form m⁷ G(5′)ppp(5′)G (“m7 GpppG”) as an initiator of transcription.

In one embodiment, the cap structure is a synthetic occurring cap structure. One example of a synthetic dinucleotide cap used in in vitro translation experiments is the Anti-Reverse Cap Analogue (“ARCA”), which is generally a modified cap analogue in which the 2′ or 3′ OH group is replaced with —OCH₃. ARCA and triple-methylated cap analogues are incorporated in the forward orientation. Chemical modification of m⁷ G at either the 2′ or 3′ OH group of the ribose ring results in the cap being incorporated solely in the forward orientation, even though the 2′ OH group does not participate in the phosphodiester bond. (Jemielity, J. et al., RNA, 2003, 9:1108-1122). The selective procedure for methylation of guanosine at N7 and 3′ O-methylation and 5′ diphosphate synthesis has been established (Kore, A. and Parmar, G. Nucleosides, Nucleotides, and Nucleic Acids, 2006, 25, 337-340, and Kore, A. R., et al. Nucleosides, Nucleotides, and Nucleic Acids 2006, 25, 307-14).

In one embodiment, the cap structure is that of β-S-ARCA, which is a structure of formula (Ic) in which B is G, B′ is 7′-methyl-G; n is 1, X₁ and X₃ is O, X₂ is S; R₁ is OH; R₂ is OCH₃; and R₃ is OH.

In one embodiment, the cap structure is that of CleanCap® 413, which is a structure of formula (Ic) in which B is A, B′ is 7′-methyl-G; n is 1, X₁, X₂ and X₃ are all O; R₁ is OH; R₂ is OH; R₃ is OCH₃, and the structure is connected at the squiggly line to G via a monophosphate intersubunit linkage. CleanCap® 413 is commercially available from TriLink Biotechnologies.

In one embodiment, the cap structure is that of CleanCap® AU, which is a structure of formula (Ic) in which B is A, B′ is 7′-methyl-G; n is 1, X₁, X₂ and X₃ are all O; R₁ is OH; R₂ is OH; R₃ is OCH₃, and the structure is connected at the squiggly line to U via a monophosphate intersubunit linkage. CleanCap® AU is commercially available from TriLink Biotechnologies.

### Production of Capped RNA

The capped RNA may be produced by any means known in the art. Typically, the RNA is produced by transcription of a corresponding DNA sequence.

In some embodiments, capped RNA is produced by in vitro transcription, originally developed by Krieg and Melton (Methods Enzymol., 1987, 155:397-415) for the synthesis of RNA using an RNA phage polymerase. Typically, these reactions include at least a phage RNA polymerase (for example, T7, T3 or SP6), a DNA template containing a phage polymerase promoter, nucleotides (in particular nucleoside triphosphates, such as ATP, CTP, GTP and UTP or modified nucleotides like N1-Me-Pseudo-UTP), and a buffer containing a salt (in particular a magnesium salt).

RNA synthesis yields may be optimized by increasing nucleotide concentrations, adjusting magnesium concentrations and by including inorganic pyrophosphatase (U.S. Pat. No. 5,256,555; Gurevich, et al., Anal. Biochem. 1991, 195 207-213; Sampson, J. R. and Uhlenbeck, O. C., Proc. Natl. Acad. Sci. USA. 1988, 85, 1033-1037; Wyatt, J. R., et al., Biotechniques, 1991, 11, 764-769. Some embodiments utilize commercial kits for the large-scale synthesis of in vitro transcripts (e.g., MEGAscript®, Ambion). The RNA synthesized in these reactions is usually characterized by a 5′ terminal nucleotide that has a triphosphate at the 5′ position of the ribose. Typically, depending on the RNA polymerase and promoter combination used, this nucleotide is a guanosine, although it can be an adenosine (see e.g., Coleman, T. M., et al., Nucleic Acids Res., 2004, 32, e14).

To synthesize a capped RNA by in vitro transcription, a cap analogue (e.g., N-7 methyl GpppG; i.e., m⁷GpppG) is included in the transcription reaction. In some embodiments, the RNA polymerase will incorporate the cap analogue as readily as any of the other nucleotides; that is, there is no bias for the cap analogue. In some embodiments, the cap analogue will be incorporated at the 5′ terminus by the enzyme guanylyl transferase.

In some embodiments using a T7, T3 and SP6 RNA polymerase, the +1 nucleotide of their respective promoters is usually a G residue and if both GTP and m⁷GpppG are present in equal concentrations in the transcription reaction, then they each have an equal chance of being incorporated at the +1 position. In some embodiments, m⁷GpppG is present in these reactions at several-fold higher concentrations than the GTP to increase the chances that a transcript will have a 5′ cap. In some embodiments, a mMESSAGE mMACHINE® kit (Cat. #1344, Ambion, Inc.) is used according to manufacturer's instructions, where it is recommended that the cap to GTP ratio be 4:1 (6 mM: 1.5 mM). In some embodiments, as the ratio of the cap analogue to GTP increases in the reaction, the ratio of capped to uncapped RNA increases proportionally.

Considerations of capping efficiency must be balanced with considerations of yield. Increasing the ratio of cap analogue to GTP in the transcription reaction produces lower yields of total RNA because the concentration of GTP becomes limiting when holding the total concentration of cap and GTP constant. Thus, the final RNA yield is dependent on GTP concentration, which is necessary for the elongation of the transcript. The other nucleotides (ATP, CTP, UTP) are present in excess.

In another embodiment, mRNA are synthesized by in vitro transcription from a plasmid DNA template encoding a gene of choice.

In one embodiment, the method comprises purifying the capped RNA. The capped RNA may be purified by any means known in the art.

In one embodiment, the capped RNA is purified using magnetic beads. As is known to the person skilled in the art, magnetic separation methods for nucleic acids involve the introduction of magnetic beads into a solution containing the RNA (typically also together with a binding buffer), followed by the application of a magnetic field (e.g. by using a permanent magnet) to separate the beads having the RNA bound thereto. The supernatant containing impurities can then be washed and the RNA eluted from the beads. Such techniques are described generally in S. Berensmeier, Appl. Microbiol. Biotech., 2006, 73, 495-504.

In one embodiment, the capped RNA is purified using tangential flow filtration (TFF). As is known to the person skilled in the art, tangential flow filtration (also known as cross-flow filtration) typically operates by passing the feed is across the filter membrane (tangentially) at positive pressure relative to the permeate side. A proportion of the material which is smaller than the membrane pore size passes through the membrane as permeate or filtrate; everything else is retained on the feed side of the membrane as retentate. The tangential motion of the bulk of the fluid across the membrane causes trapped particles on the filter surface to be rubbed off.

Step (b)—Enzymatic Hydrolysis of Capped RNA

According to step (b) of the method of the present invention, the capped RNA is hydrolysed to produce hydrolysis products comprising a capped product comprising dinucleotides, and an uncapped product comprising nucleotides.

The capped RNA is hydrolysed by contacting it with a nuclease. The precise nature of the nuclease is not limited, provided that it is capable of hydrolysing the capped RNA to produce a capped product comprising dinucleotides and an uncapped product comprising nucleotides. Typically, the nuclease is a protein.

In one embodiment, the nuclease is incapable of hydrolysing nucleotides into nucleosides.

In step (b), the nuclease may be present as part of an enzyme composition containing additional enzymes. In this embodiment, typically the nuclease comprise more than 50% by weight, such as more than 60% by weight, such as more than 70% by weight, such as more than 80% by weight, such as more than 90% by weight, such as more than 95% by weight, such as more than 96% by weight, such as more than 97% by weight, such as more than 98% by weight, such as more than 99% by weight, such as more than 99.5% by weight, such as more than 99.7% by weight, such as more than 99.9% by weight, such as more than 99.99% by weight, of the total weight of the uncapped hydrolysis products.

Step (b) is carried out in the absence of a nucleic acid having a base sequence complementary to the sequence of the capped RNA. In one embodiment, step (b) is carried out in the absence of a DNA having a base sequence complementary to the sequence of the capped RNA. In contrast to the method of WO2014/152659, in which a nuclease is used in combination with a complementary oligonucleotide probe to hydrolyse the RNA, use of the nuclease as the sole hydrolysing agent avoids the need to prepare and test a specific complementary probe for every different RNA which is to be quantified by means of the assay method. This makes the method more straightforward to use and applicable to any RNA sequence.

In one embodiment, the nuclease is the sole agent which hydrolyses the RNA. In contrast to the method used in Muthmann et al. and the method of WO2017/149139, in which a nuclease is used in combination with an alkaline phosphatase and a phosphodiesterase to fully hydrolyse the RNA into nucleosides, use of the nuclease as the sole hydrolysing agent, thereby limiting the extent of the hydrolysis to nucleotides, allows the method to be carried out using a single enzyme rather than multiple enzymes, thereby simplifying the method by reducing the number of enzymatic steps and lessening or eliminating the risk of cross-reactivity between the enzymes.

In one embodiment, step (b) is carried out in the absence of a PDE1. In one embodiment, step (b) is carried out in the absence of a snake venom phosphodiesterase. In one embodiment, step (b) is carried out in the absence of a phosphatase. In one embodiment, step (b) is carried out in the absence of an alkaline phosphatase.

In one embodiment, the nuclease used in step (b) is nuclease P1 or nuclease S1. In one embodiment, the nuclease used in step (b) is nuclease P1.

In one embodiment, step (b) is carried out at a nuclease concentration of 10 to 70 μmol/L, preferably 20 to 35 μmol/L.

In one embodiment, step (b) is carried out at a temperature of room temperature to 60° C. In one embodiment, step (b) is carried out at a temperature of 30 to 55° C. In one embodiment, step (b) is carried out at a temperature of 37° C. In one embodiment, step (b) is carried out at a temperature of 50° C.

In one embodiment, step (b) is carried out for a time of 30 minutes to 48 hours. In one embodiment, step (b) is carried out for a time of 1 hour to 36 hours. In one embodiment, step (b) is carried out for a time of 2 hours to 30 hours. In one embodiment, step (b) is carried out for a time of 3 hours to 24 hours.

In one embodiment, step (b) is carried out at a pH of 4 to 6. In one embodiment, step (b) is carried out at a pH of 4.3 to 5.5. In one embodiment, step (b) is carried out at a pH of 4.5. In one embodiment, step (b) is carried out at a pH of 5.3.

### Capped Hydrolysis Product

The hydrolysis of RNA according to the method of the present invention results in a capped RNA product.

In one embodiment, the capped hydrolysis product comprises a dinucleotide. In one embodiment, the capped hydrolysis product consists essentially of a dinucleotide. In one embodiment, the capped hydrolysis product is a dinucleotide. In one embodiment, the capped hydrolysis product consists of a dinucleotide. The dinucleotide is as defined and exemplified above.

In one embodiment, the dinucleotide comprise more than 50% by weight, such as more than 60% by weight, such as more than 70% by weight, such as more than 80% by weight, such as more than 90% by weight, such as more than 95% by weight, such as more than 96% by weight, such as more than 97% by weight, such as more than 98% by weight, such as more than 99% by weight, such as more than 99.5% by weight, such as more than 99.7% by weight, such as more than 99.9% by weight, such as more than 99.99% by weight, of the total weight of the capped hydrolysis products.

In one embodiment, the capped hydrolysis product has a structure of formula (II):

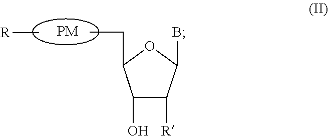

wherein:
PM is a mono- or polyphosphate moiety containing 1, 2, 3, 4, 5, 6, 7, 8, 9 or 10 phosphate units, the oxo (═O) groups in any of the phosphate units being optionally replaced with a thio (═S) group;
R is an end-cap moiety;
B is a nucleobase, optionally alkylated on a nitrogen atom by a C₁₋₄ alkyl group; and
R′ is selected from OH, O(C₁₋₄ alkyl), and halogen.

In formula (II), R may represent any group capable of allowing the cap to perform the above-mentioned function of binding to the cap-binding complex and EIF4E enabling the RNA to undergo translation during protein synthesis and/or protecting the RNA from degradation via 5′-3′ exonucleases.

In one embodiment, the capped hydrolysis product has a structure of formula (IIa):

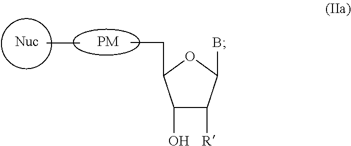

wherein:
PM is a mono- or polyphosphate moiety containing 1, 2, 3, 4, 5, 6, 7, 8, 9 or 10 phosphate units, the oxo (═O) groups in any of the phosphate units being optionally replaced with a thio (═S) group;
Nuc is a nucleoside or nucleoside analogue;
B is a nucleobase, optionally alkylated on a nitrogen atom by a C₁₋₄ alkyl group; and
R′ is selected from OH, O(C₁₋₄ alkyl), and halogen.

In formulae (II) and (IIa), PM is preferably a monophosphate (1 phosphate unit), diphosphate (2 phosphate units), triphosphate (3 phosphate units) or tetraphosphate (4 phosphate units). Any one or all of the oxo (═O) groups in the phosphate units (preferably only 1 oxo group in 1 phosphate unit) may be replaced with a thio (═S) group.

In one embodiment of formula (IIa), Nuc is a nucleoside, which may be a ribonucleoside or deoxyribonucleoside (as defined above).

In another embodiment of formula (IIa), Nuc is a nucleoside analogue, as defined above. The nucleoside analogue may comprise a locked nucleic acid (LNA) moiety, a peptide nucleic acid (PNA) moiety, an unlocked nucleic acid (UNA) moiety or a morpholino moiety, as defined above.

In one embodiment, the capped hydrolysis product has a structure of formula (IIb):

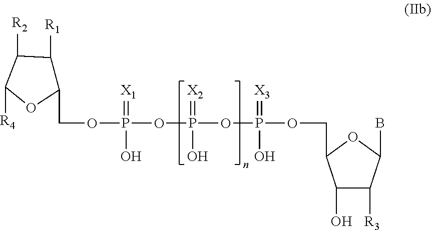

or a salt thereof, wherein:
B is a nucleobase, optionally alkylated on a nitrogen atom by a C₁₋₄ alkyl group;
R₁ is selected from OH, O(C₁₋₄ alkyl), and halogen;
R₂ is selected from H, OH, and O(C₁₋₄ alkyl), and halogen;
R₃ is selected from OH, O(C₁₋₄ alkyl), and halogen;
R₄ is H, OH, O(C₁₋₄ alkyl), halogen, or a nucleobase, optionally alkylated on a nitrogen atom by a C₁₋₄ alkyl group;
n is 1, 2 or 3; and
X₁, each X₂, and X₃, are each independently O or S.

In one embodiment of formula (IIb), R₄ is OH. In one embodiment of formula (IIb), R₄ is a nucleobase, optionally alkylated on a nitrogen atom by a C₁₋₄ alkyl group.

In one embodiment, the capped hydrolysis product has a structure of formula (IIc):

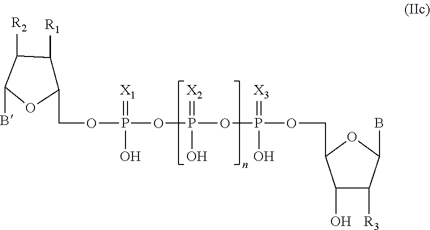

or a salt thereof, wherein:
B and B′ are each independently nucleobases, each optionally alkylated on a nitrogen atom by a C₁₋₄ alkyl group;
R₁ is selected from OH, O(C₁₋₄ alkyl), and halogen;
R₂ is selected from H, OH, and O(C₁₋₄ alkyl), and halogen;
R₃ is selected from OH, O(C₁₋₄ alkyl), and halogen;
n is 1, 2 or 3; and
each X and X′ is independently O or S.

In one embodiment of formula (IIc), B is selected from adenine (“A”), guanine (“G”), cytosine (“C”), or uracil (“U”), each optionally alkylated on a nitrogen atom by a C₁₋₄ alkyl group, such as by a methyl group. In one embodiment of formula (IIc), B is G, optionally methylated on the nitrogen at the 7′-position.

In one embodiment of either formula (IIb) or (IIc), B′ is selected from adenine (“A”), guanine (“G”), cytosine (“C”), or uracil (“U”), each optionally alkylated on a nitrogen atom by a C₁₋₄ alkyl group, such as by a methyl group. In one embodiment of either formula (IIa) or (IIb), B′ is G, optionally methylated on the nitrogen at the 7′-position.

In one embodiment of either formula (IIb) or (IIc), R₁ is OH or OCH₃.

In one embodiment of either formula (IIb) or (IIc), R₂ is H, OH, or OCH₃.

In one embodiment of either formula (IIb) or (IIc), R₃ is OH or OCH₃.

In one embodiment of either formula (IIb) or (IIc), n is 1.

In one embodiment of either formula (IIb) or (IIc), X₁ is O. In one embodiment of either formula (IIb) or (IIc), X₃ is O. In one embodiment of either formula (IIb) or (IIc), X₁ is S. In one embodiment of either formula (IIb) or (IIc), X₃ is S. In one embodiment of either formula (IIb) or (IIc), each X₂ is O. In one embodiment of either formula (IIb) or (IIc), each X₂ is S.

In one embodiment of formula (IIc), B and B′ are both G, each optionally methylated on the nitrogen at the 7′-position. In one embodiment of formula (IIc), B is G, and B′ is 7′-methyl-G.

In one embodiment of either formula (IIb) or (IIc), n is 1, X₁ and X₃ are O, and X₂ is O. In one embodiment of either formula (IIb) or (IIc), n is 1, X₁ and X₃ are O, and X₂ is S.

In one embodiment, the capped hydrolysis products are selected from the group consisting of m⁷G(5′)ppp(5′)N, m⁷G(5′)ppp(5′)-(cap 0), m⁷G(5′)ppp(5′) Nm-(cap 1), m⁷G(5′)ppp(5′)G, ARCA, β-S-ARCA, wherein G is guanosine, p is a phosphate residue, N is any nucleoside, and Nm is a nucleoside having a 2′-methyl group.

In one embodiment, the capped hydrolysis product is that of the dinucleotide resulting from the hydrolysis of an RNA capped with β-S-ARCA, i.e. a structure of formula (IIc) in which B is G, B′ is 7′-methyl-G; n is 1, X₁ and X₃ is O, X₂ is S; R₁ is OH; R₂ is OCH₃; and R₃ is OH.

In one embodiment, the capped hydrolysis product is that of the dinucleotide resulting from the hydrolysis of an RNA capped with CleanCap® 413, i.e. a structure of formula (IIc) in which B is A, B′ is 7′-methyl-G; n is 1, X₁, X₂ and X₃ are all O; R₁ is OH; R₂ is OH; and R₃ is OCH₃.

In one embodiment, the capped hydrolysis product is that of the dinucleotide resulting from the hydrolysis of an RNA capped with CleanCap® AU, i.e. a structure of formula (IIc) in which B is A, B′ is 7′-methyl-G; n is 1, X₁, X₂ and X₃ are all O; R₁ is OH; R₂ is OH; and R₃ is OCH₃.

### Uncapped Hydrolysis Product

The hydrolysis of RNA according to the method of the present invention also results in an uncapped RNA product.

In one embodiment, the uncapped hydrolysis product comprises a nucleotide. In one embodiment, the uncapped hydrolysis product consists essentially of a nucleotide. In one embodiment, the uncapped hydrolysis product is a dinucleotide. In one embodiment, the uncapped hydrolysis product consists of a dinucleotide.

The nucleotide is as defined and exemplified above, i.e. a phosphate ester of a nucleoside. The phosphate group may be present at any oxygen on the sugar portion of the nucleotide. Typically, the phosphate group is present on the 3′-position or the 5′-position, preferably the 5′-position. The phosphate group may comprise any number of phosphate units, typically 1, 2, 3, 4, 5, 6, 7, 8, 9, or 10 phosphate units. Preferably, the phosphate group is a monophosphate (1 phosphate unit), diphosphate (2 phosphate units) or triphosphate (3 phosphate units).

In one embodiment, nucleotides comprise more than 50% by weight, such as more than 60% by weight, such as more than 70% by weight, such as more than 80% by weight, such as more than 90% by weight, such as more than 95% by weight, such as more than 96% by weight, such as more than 97% by weight, such as more than 98% by weight, such as more than 99% by weight, such as more than 99.5% by weight, such as more than 99.7% by weight, such as more than 99.9% by weight, such as more than 99.99% by weight, of the total weight of the uncapped hydrolysis products.

In one embodiment, the nucleotide comprises, consists essentially of or consists of a nucleoside monophosphate. In one embodiment, the nucleotide comprises, consists essentially of or consists of a nucleoside 5′-monophosphate. In one embodiment, the nucleotide comprises, consists essentially of or consists of a nucleoside 3′-monophosphate.

In one embodiment, the nucleotide comprises, consists essentially of or consists of a nucleoside diphosphate. In one embodiment, the nucleotide comprises, consists essentially of or consists of a nucleoside 5′-diphosphate. In one embodiment, the nucleotide comprises, consists essentially of or consists of a nucleoside 3′-diphosphate.

In one embodiment, the nucleotide comprises, consists essentially of or consists of a nucleoside triphosphate. In one embodiment, the nucleotide comprises, consists essentially of or consists of a nucleoside 5′-triphosphate. In one embodiment, the nucleotide comprises, consists essentially of or consists of a nucleoside 3′-triphosphate.

In one embodiment, the nucleotide comprises, consists essentially of or consists of a guanosine monophosphate. In one embodiment, the nucleotide comprises, consists essentially of or consists of a guanosine 5′-monophosphate. In one embodiment, the nucleotide comprises, consists essentially of or consists of a guanosine 3′-monophosphate.

In one embodiment, the nucleotide comprises, consists essentially of or consists of a guanosine diphosphate. In one embodiment, the nucleotide comprises, consists essentially of or consists of guanosine 5′-diphosphate. In one embodiment, the nucleotide comprises, consists essentially of or consists of guanosine 3′-diphosphate.

In one embodiment, the nucleotide comprises, consists essentially of or consists of a guanosine triphosphate. In one embodiment, the nucleotide comprises, consists essentially of or consists of guanosine 5′-triphosphate. In one embodiment, the nucleotide comprises, consists essentially of or consists of guanosine 3′-triphosphate.

In one embodiment, the nucleotide comprises, consists essentially of or consists of an adenosine monophosphate. In one embodiment, the nucleotide comprises, consists essentially of or consists of a adenosine 5′-monophosphate. In one embodiment, the nucleotide comprises, consists essentially of or consists of a adenosine 3′-monophosphate.

In one embodiment, the nucleotide comprises, consists essentially of or consists of an adenosine diphosphate. In one embodiment, the nucleotide comprises, consists essentially of or consists of adenosine 5′-diphosphate. In one embodiment, the nucleotide comprises, consists essentially of or consists of adenosine 3′-diphosphate.

In one embodiment, the nucleotide comprises, consists essentially of or consists of an adenosine triphosphate. In one embodiment, the nucleotide comprises, consists essentially of or consists of adenosine 5′-triphosphate. In one embodiment, the nucleotide comprises, consists essentially of or consists of adenosine 3′-triphosphate.

In one embodiment, the nucleotide comprises, consists essentially of or consists of a uridine monophosphate. In one embodiment, the nucleotide comprises, consists essentially of or consists of uridine 5′-monophosphate. In one embodiment, the nucleotide comprises, consists essentially of or consists of uridine 3′-monophosphate.

In one embodiment, the nucleotide comprises, consists essentially of or consists of a uridine diphosphate. In one embodiment, the nucleotide comprises, consists essentially of or consists of uridine 5′-diphosphate. In one embodiment, the nucleotide comprises, consists essentially of or consists of uridine 3′-diphosphate.

In one embodiment, the nucleotide comprises, consists essentially of or consists of a uridine triphosphate. In one embodiment, the nucleotide comprises, consists essentially of or consists of uridine 5′-triphosphate. In one embodiment, the nucleotide comprises, consists essentially of or consists of uridine 3′-triphosphate.

In one embodiment, the nucleotide comprises, consists essentially of or consists of a cytidine monophosphate. In one embodiment, the nucleotide comprises, consists essentially of or consists of cytidine 5′-monophosphate. In one embodiment, the nucleotide comprises, consists essentially of or consists of cytidine 3′-monophosphate.

In one embodiment, the nucleotide comprises, consists essentially of or consists of a cytidine diphosphate. In one embodiment, the nucleotide comprises, consists essentially of or consists of cytidine 5′-diphosphate. In one embodiment, the nucleotide comprises, consists essentially of or consists of cytidine 3′-diphosphate.

In one embodiment, the nucleotide comprises, consists essentially of or consists of a cytidine adenosine triphosphate. In one embodiment, the nucleotide comprises, consists essentially of or consists of cytidine 5′-triphosphate. In one embodiment, the nucleotide comprises, consists essentially of or consists of cytidine 3′-triphosphate.

In one embodiment, the uncapped hydrolysis products include guanosine 5′-triphosphate (GTP) and the method includes determining the amount of GTP.

In one embodiment, the uncapped hydrolysis products include adenosine 5′-triphosphate (ATP) and the method includes determining the amount of ATP.

Step (c)—Chromatographic Separation

Step (c) of the method of the present invention comprises separating the hydrolysis products by chromatography. Generally, the term “chromatography” refers to a technique for separation of mixtures in which, typically, the mixture is dissolved in a fluid called the “mobile phase,” or “eluent”, which carries it through a structure holding another material called the “stationary phase.”

Chromatography may be carried out according to a wide range of possible techniques, which are generally well known to those skilled in the art. Any chromatography method may be used provided that it is capable of being coupled to the mass spectrometry method used in step (d) as below.

The chromatography technique may be classified by the physical state of the mobile phase. In one embodiment, the chromatography method used in step (c) is liquid chromatography (i.e. wherein the mobile phase is a liquid). In one embodiment, the chromatography method used in step (c) is gas chromatography (i.e. wherein the mobile phase is a gas).

When the chromatography method used in step (c) is liquid chromatography, methods in which the stationary phase is more polar than the mobile phase (e.g., toluene as the mobile phase, silica as the stationary phase) are termed normal phase liquid chromatography (NPLC) and the opposite (e.g., water-methanol mixture as the mobile phase and C18 (octadecylsilyl) as the stationary phase) is termed reversed phase liquid chromatography (RPLC).

When the chromatography method used in step (c) is liquid chromatography, in one embodiment the chromatography used in step (c) is high-performance liquid chromatography (HPLC). As is known to the person skilled in the art, HPLC is a technique in analytical chemistry used to separate, identify, and quantify each component in a mixture. It relies on pumps to pass a pressurized liquid solvent containing the sample mixture through a column filled with a solid adsorbent material. Each component in the sample interacts slightly differently with the adsorbent material, causing different flow rates for the different components and leading to the separation of the components as they flow out of the column. Typically, this is carried out at a pressure of around 50-600 bar. Typically this is carried out in a column of 1 to 10 mm diameter, preferably 2 to 5 mm diameter. Typically this is carried out in a column of 10 to 500 mm length, preferably 30 to 250 mm diameter length. Typically this is carried out using adsorbent particles of an average particle size between 1 to 100 μm, preferably 2 to 50 μm.

In one embodiment the chromatography used in step (c) is ultra performance liquid chromatography. Typically, this is carried out at a pressure of around 400-1200 bar. Typically, this is carried out in a column of 0.5 to 5 mm diameter, preferably 1 to 4 mm diameter. Typically, this is carried out in a column of 5 to 300 mm length, preferably 10 to 250 mm length. Typically, this is carried out using adsorbent particles of an average particle size of 0.1 to 10 μm, preferably 0.5 to 3 μm.

The chromatography technique may also be classified by the separation mechanism. In one embodiment, the chromatography used in step (c) is hydrophilic interaction chromatography. In one embodiment, the chromatography used in step (c) is ion-exchange chromatography. In one embodiment, the chromatography used in step (c) is reverse-phase chromatography. In one embodiment, the chromatography used in step (c) is size exclusion chromatography.

In one embodiment, the chromatography technique used in step (c) is reverse-phase liquid chromatography (RPC). As is known to the person skilled in the art, the term “reverse-phase chromatography” refers to any liquid chromatography procedure in which the mobile phase is significantly more polar than the stationary phase.

In one embodiment, the chromatography technique used in step (c) is ion-exchange chromatography. As is known to the person skilled in the art, the term “ion exchange chromatography” (or “ion chromatography)” refers to a chromatography technique which separates ions and polar molecules based on their affinity to the ion exchanger. Ion-exchange chromatography separates molecules based on their respective charged groups. Ion-exchange chromatography retains analyte molecules on the column based on coulombic (ionic) interactions. The ion exchange chromatography matrix consists of positively and negatively charged ions. Molecules undergo electrostatic interactions with opposite charges on the stationary phase matrix. The stationary phase consists of an immobile matrix that contains charged ionizable functional groups or ligands. The stationary phase surface displays ionic functional groups (R-X) that interact with analyte ions of opposite charge. To achieve electroneutrality, these inert charges couple with exchangeable counterions in the solution. Ionizable molecules that are to be purified compete with these exchangeable counterions for binding to the immobilized charges on the stationary phase. These ionizable molecules are retained or eluted based on their charge. Initially, molecules that do not bind or bind weakly to the stationary phase are first to wash away. Altered conditions are needed for the elution of the molecules that bind to the stationary phase. The concentration of the exchangeable counterions, which competes with the molecules for binding, can be increased or the pH can be changed. A change in pH affects the charge on the particular molecules and, therefore, alters binding. The molecules then start eluting out based on the changes in their charges from the adjustments. Further such adjustments can be used to release the protein of interest. Additionally, concentration of counterions can be gradually varied to separate ionized molecules. This type of elution is called gradient elution. On the other hand, step elution can be used in which the concentration of counterions are varied in one step.

Ion exchange chromatography may be further subdivided into cation exchange chromatography and anion-exchange chromatography. Positively charged molecules bind to cation exchange resins while negatively charged molecules bind to anion exchange resins. The ionic compound consisting of the cationic species M⁺ and the anionic species B⁻ can be retained by the stationary phase. Cation exchange chromatography retains positively charged cations because the stationary phase displays a negatively charged functional group: Anion exchange chromatography retains anions using positively charged functional group:

In one embodiment, the chromatography technique used in step (c) is hydrophilic interaction chromatography (or hydrophilic interaction liquid chromatography, HILIC). As is known to the person skilled in the art, the term “hydrophilic interaction chromatography” denotes a technique in which the mobile phase is hydrophobic and the stationary phase is hydrophilic, such that the order of elution is typically the opposite of that obtained with reverse-phase chromatography—see A. J. Alpert, J. Chromatography A, 1990, 499, 177-196. As indicated above, determination of the analytes using HILIC in combination with mass spectroscopy using a triple quadrupole mass spectrometer is particularly advantageous for the quantitation of nucleotides, as HILIC makes it possible to retain and separate very polar small analytes such as nucleotides. The stationary phase may be an unbonded silica, silanol or diol bonded phase; an amino or anionic bonded phase, an amide bonded phase, a cationic bonded phase, or a zwitterionic bonded phase. Preferably the stationary phase is an amide bonded phase.

In one embodiment, the chromatography technique used in step (c) is ion interaction chromatography (also known as ion-pair chromatography). As is known to the person skilled in the art, this term denotes a reversed phase technique in which charged substances are mixed with ion pairing reagents (IPR) added to the mobile phase, the analyte typically combining with its reciprocal ion in the IPR. The formation of this pair affects the interaction of the pair with the mobile phase and the stationary phase of the column, thus permitting the separation of different ion pairs.

When the chromatography used in step (c) is liquid chromatography, the mobile phase (eluent) may be any suitable liquid known in the art. Suitable examples include water, C1-4 alcohols such as methanol, ethanol and isopropanol, C1-4 halogenated alcohols such as hexafluoroisopropanol, aprotic solvents miscible with water (e.g., nitriles such as acetonitrile, and ethers, especially cyclic ethers such as tetrahydrofuran and 1,4-dioxane), and mixtures of any thereof. In one embodiment, the mobile phase used in the liquid chromatography is a mixture of water and acetonitrile. In one embodiment, the mobile phase used in the liquid chromatography is hexafluoroisopropanol.

In one embodiment, the mobile phase used in the liquid chromatography includes a buffer. As is known to the person skilled in the art, a buffer solution is an aqueous solution consisting of a mixture of a weak acid and its conjugate base, or vice versa. Its pH changes very little when a small amount of strong acid or base is added to it. The buffer may be any suitable buffer known in the art. Suitable examples include citric acid/citrate buffers, acetic acid/acetate buffers, phosphate buffers, borate buffers, ammonia/ammonium salt buffers, carbonate/hydrogen carbonate-based buffers, bicine (2-(bis(2-hydroxyethyl)amino) acetic acid), Tris (tris(hydroxymethyl)aminomethane, or 2-amino-2-(hydroxymethyl) propane-1,3-diol), tricine (N-[tris(hydroxymethyl)methyl]glycine), TAPSO (3-[N-tris(hydroxymethyl)-methylamino]-2-hydroxypropanesulfonic acid), HEPES (4-(2-hydroxyethyl)-1-piperazineethanesulfonic acid), TES (2-[[1,3-dihydroxy-2-(hydroxymethyl) propan-2-yl]amino]ethanesulfonic acid), MOPS (3-(N-morpholino) propanesulfonic acid), PIPES (piperazine-N,N′-bis(2-ethanesulfonic acid)) and MES (2-(N-morpholino) ethanesulfonic acid). In one embodiment, the buffer is ammonium carbonate.

When the chromatography used in step (c) is liquid chromatography, in one embodiment, step (c) is carried out at a temperature of room temperature to 90° C. In one embodiment, step (c) is carried out at a temperature of 30 to 70° C. In one embodiment, step (c) is carried out at a temperature of 55° C.

When the chromatography used in step (c) is liquid chromatography, in one embodiment, step (c) is carried out using a stationary phase, which may be any fine adsorbent solid. Typical examples include silica and alumina.

When the chromatography used in step (c) is liquid chromatography, in one embodiment, step (c) is carried out at a pH of 2 to 11, preferably 8 to 10.

When the chromatography used in step (c) is liquid chromatography, in one embodiment, step (c) is carried out at a flow rate of 0.1 to 2 ml/min, preferably 0.3 to 1.0 ml/min.

Step (d)—Mass Spectrometry

Step (d) of the method of the present invention comprises determining the concentrations of the hydrolysis products by mass spectrometry, thereby quantifying RNA capping efficiency.

As is known to the person skilled in the art, a mass spectrometer typically consists of three components: an ion source, a mass analyzer, and a detector. The ionizer converts a portion of the sample into ions. As detailed below, there are a wide variety of ionization techniques, depending on the phase (solid, liquid, gas) of the sample and the efficiency of various ionization mechanisms for the unknown species. The mass spectrometer also typically comprises an extraction system which removes ions from the sample, which are then targeted through the mass analyzer and onto the detector. The difference in mass-to-charge (m/z) of the fragments allows the mass analyzer to sort the ions by their mass-to-charge ratio. Finally, the detector measures the value of an indicator quantity and thus provides data for calculating the abundances of each ion present.

In a typical mass spectrometry procedure, the first step comprises ionization of a sample. In one embodiment, the ionization comprises electron ionization (EI), which comprises bombarding the sample with electrons. In another embodiment, the ionization comprises chemical ionization (CI), according to which ions are produced through the collision of the analyte with ions of a reagent gas that are present in the ion source (examples of suitable reagent gases include methane, ammonia, and isobutane). In another embodiment, the ionization comprises Atmospheric Pressure Chemical Ionisation (APCI). In another embodiment, the ionization comprises Atmospheric Pressure Photon Ionization (APPI).

When the ionization is electron ionization, this typically results in a mass ion having the same mass (M) as the parent molecule but a charge (M⁺ or M⁻). When the ionization is chemical ionization, this typically results in a mass ion having a mass of the parent molecule and the chemical species used to ionize the molecule, well known examples including [M+H]⁺, [M−H]⁻, [M+NH₄]⁺ and [M+Na]⁺. Such a molecular ion is also referred to in this specification as a “pseudo molecular ion”.

In another embodiment, the ionization comprises electrospray ionization (ESI), in which the liquid containing the analyte(s) of interest is dispersed by electrospray into a fine aerosol. In another embodiment, the ionization comprises matrix-assisted laser desorption/ionization (MALDI), which typically comprises a three-step process, as follows: (1) mixing the sample is a suitable matrix material and applying it to a surface, typically a metal plate; (2) irradiating the sample, typically with a pulsed laser, thereby triggering ablation and desorption of the sample and matrix material; and (3) ionization of the analyte molecules by being protonated or deprotonated in the hot plume of ablated gases, allowing the ions to be accelerated into the mass spectrometer used to analyse them. These ionization techniques are well known to the person skilled in the art. Ionization, in particular electron ionization, may cause some of the sample's molecules to break into charged fragments.

Following ionization, the ions produced in the first step are then separated according to their mass-to-charge (m/z) ratio in the mass analyzer. This is typically carried out by one or more of the following mass to charge separation techniques: by quadrupole electric fields as used in quadrupole mass spectrometers, by ion trap quadrupole electric fields as used by ion trap mass spectrometers, by longitudinal ion travelling time as used by time of flight mass spectrometers and by electric and/or magnetic field deflection as traditionally used by electric and magnetic sector mass spectrometers. This last technique involves accelerating the ions and subjecting them to an electric or magnetic field, such that the electric or magnetic field causes the ions to be deflected. Ions of the same mass-to-charge ratio will undergo the same amount of deflection.

Following separation, the ions are detected. Typically, the detector records either the charge induced or the current produced when an ion passes by or hits a surface. In a scanning instrument, the signal produced in the detector during the course of the scan versus where the instrument is in the scan will produce a mass spectrum, a record of ions as a function of m/z.

According to the present invention, the mass spectrometry step (d) is used in tandem with a chromatographic separation technique in step (c). In one embodiment, the chromatographic technique is gas chromatography, the combination technique being known as gas chromatography-mass spectrometry (GC/MS, GCMS or GC-MS). As is known to the person skilled in the art, in this technique, a gas chromatograph is used to separate different compounds. This stream of separated compounds is fed into the mass spectrometer for ionization, mass analysis and detection as described above.

In one embodiment, the chromatographic technique is liquid chromatography, the combination technique being known as liquid chromatography-mass spectrometry (LC/MS, LCMS or LC-MS). As described in relation to step (c) above, and is generally known to the person skilled in the art, this technique separates compounds chromatographically using a liquid mobile phase. Typically, the liquid phase is a mixture of water and organic solvents. The stream of separated compounds is then fed into the mass spectrometer for ionization, mass analysis and detection as described above.

In one embodiment, the mass spectrometry is direct sampling mass spectrometry. As is known to the person skilled in the art, this technique involves the introduction of a sampling probe containing the sample to be analysed directly into the ionisation chamber of the mass spectrometer. The sample may be solid, liquid or gas, preferably solid.

In one embodiment, the mass spectrometry is infusion sampling mass spectrometry. As is known to the person skilled in the art, this technique involves the introduction of the sample to be analysed into the mass spectrometer by spraying a liquid containing the sample into the mass spectrometer.

The mass spectrometry used in step (d) is tandem mass spectrometry. Tandem mass spectrometry, also known as MS/MS, MS² or MSⁿ (where n is at least 2, preferably 2 to 10, more preferably 2 to 5, even more preferably 2 or 3, most preferably 2) involves multiple steps of mass spectrometry selection, with some form of fragmentation occurring in between the steps. Tandem mass spectrometry is especially preferred as the mass spectrometry method of the present invention, as particularly although not exclusively when coupled to liquid chromatography, the analytes can be determined with high selectivity, flexibility and sensitivity.

Typically, tandem mass spectrometry involves the following steps:
(i) Ionization of a sample to produce ions. The ionization may be carried out using any of the ionization techniques generally described above, in particular Electron Impact (EI), Electrospray Ionization (ESI), Secondary Electrospray Ionization (SESI), Desorption Electrospray Ionization (DESI), Easy Ambient Sonic Spray Ionisation (EASI), Extractive Electrospray Ionization (EESI), Neutral Desorption Electrospray Ionization (ND-ESI), Jet Desorption Electrospray Ionization (JEDI), Liquid Extraction Surface Analysis (LESA), Surface Activated Chemical Ionization (SACI), Atmospheric Pressure Chemical Ionization (APCI), Atmospheric Pressure Photon Ionization (APPI), Direct Analysis in Real Time (DART), or Matrix Assisted Laser Desorption Ionization (MALDI).
(ii) Separating the ions according to their mass-to-charge ratio to produce one or more precursor ions. The separation is carried out as generally described above.
(iii) Fragmentation of the one or more separated precursor ions to yield a production. There are many methods used to fragment the ions and these can result in different types of fragmentation and thus different information about the structure and composition of the molecule. In one embodiment, the fragmentation method comprises collision-induced dissociation. Typically, this method involves the collision of an ion with a neutral atom or molecule in the gas phase and subsequent dissociation of the ion. In one embodiment, the fragmentation method comprises an electron impact capture and/or transfer method. Typically, this method uses the energy released when an electron is transferred to or captured by a multiply charged ion to induce fragmentation. Examples of electron capture and/or transfer methods used to induce fragmentation include electron capture dissociation, electron transfer dissociation, negative electron transfer dissociation, electron-detachment dissociation, and charge transfer dissociation. In one embodiment, the fragmentation method comprises photodissociation. Typically, in this method the energy required for dissociation can be added by photon absorption. Examples of photodissociation methods include infrared multi-photon dissociation, blackbody infrared radiative dissociation or surface induced dissociation. In another embodiment, the fragmentation technique comprises in-source fragmentation (i.e. fragmentation in the ionization chamber) in which the ionization process is sufficiently violent to leave the resulting ions with sufficient internal energy to fragment within the mass spectrometer (e.g. by electron impact, Chemical Ionization or “accelerated ion dissociation”). All of these techniques are well known to the person skilled in the art.
(iv) Separating the product ions obtained from the fragmentation process according to their mass-to-charge ratio. The separation is typically carried out as generally described above.
(v) Detection of the separated ions. The detection is typically carried out as generally described above.

In one embodiment, the tandem mass spectrometry is ion trap mass spectrometry. As is known to the person skilled in the art, a quadrupole ion trap is a type of ion trap that uses dynamic electric fields to trap charged particles.

The tandem mass spectrometry used in step (d) is triple quadrupole mass spectrometry (TQMS). As is known to the person skilled in the art, a triple quadrupole mass spectrometer is a tandem mass spectrometer consisting of two quadrupole mass analyzers in series, with a (non-mass-resolving) radio frequency-only quadrupole between them to act as a cell for collision-induced dissociation. TQMS allows detection at a higher sensitivity than other tandem mass spectrometry methods.

In one embodiment, the triple quadrupole mass spectrometry used in step (d) uses a multiple reaction monitoring (B) method. MRM allows detection at a higher sensitivity and selectivity than other tandem mass spectrometry methods. As is known to the person skilled in the art, a triple quadrupole mass spectrometer can be used in different scan modes. A full scan mode is a single stage scan type that provides a full mass spectrum of each analyte. The mass analyzer is scanned from the low mass to the high mass of the user's defined mass range. A product ion scan involves the selection of ions of one mass-to-charge ratio (the parent ions). Then, those ions are subjected to collisions with collision gas in collision cell. The collisions of the parent ions cause them to fragment and produce product ions. A precursor ion scan scans precursor ions in Q1 and selects certain fragment ions in Q3. All collision induced dissociation carried out in Q2. A Neutral loss scan scans all ions in Q1 and selects ions with neutral loss in Q3. Selected Ion Monitoring (SIM) is a single stage technique in which a desired ion or set of ions is monitored. Selected Reaction Monitoring (SRM) or multiple reaction monitoring (MRM) is a two stage (MS/MS) technique in which parent ion and product ion pairs are monitored. In MRM mode, analytes can be measured in a complex mixture because the sample matrix (other sample components) is mostly removed through this two-step filtering mechanism.

In one embodiment, the tandem mass spectrometry is quadrupole time of flight mass spectrometry. As is known to the person skilled in the art, a quadrupole time-of-flight mass spectrometer is a triple quadrupole mass spectrometer, as described above, with the final quadrupole replaced by a time-of-flight device. As is known to the person skilled in the art, time-of-flight mass spectrometry (TOFMS) is a method of mass spectrometry in which the mass-to-charge ratio (m/z) of an ion is determined via a time measurement. The technique involves acceleration of the ions by an electric field of known strength. This acceleration results in an ion having the same kinetic energy as any other ion that has the same charge. The velocity of the ion depends on the mass-to-charge ratio. The time that it subsequently takes for the particle to reach a detector at a known distance is measured. This time will depend on the mass-to-charge ratio of the particle, heavier particles reaching lower speeds. From this time and the known experimental parameters, the user can determine the mass-to-charge ratio of the ion.

In one embodiment, the tandem mass spectrometry is Quadrupole Ion Trap mass spectrometry. In one embodiment, the tandem mass spectrometry is Quadrupole-Time of Flight mass spectrometry. In one embodiment, the tandem mass spectrometry is Ion Mobility-Quadrupole Ion Trap-Time of Flight mass spectrometry. In one embodiment, the tandem mass spectrometry is Quadrupole-Orbitrap mass spectrometry. In one embodiment, the tandem mass spectrometry is Quadrupole Ion Trap mass spectrometry. In one embodiment, the tandem mass spectrometry is Ion Mobility Spectrometer-Quadrupole Ion Trap mass spectrometry. In one embodiment, the tandem mass spectrometry is Quadrupole-Orbitrap Mass spectrometry. In one embodiment, the tandem mass spectrometry is a Triple-Quadrupole-Orbitrap mass spectrometry. In one embodiment, the tandem mass spectrometry is Quadrupole Ion Trap-Orbitrap mass spectrometry. In one embodiment, the tandem mass spectrometry is Time of Flight, Ion Trap-Fourier Transform mass spectrometry. Details of these techniques are known to the person skilled in the art.

In one embodiment, the tandem mass spectrometry is secondary electrospray ionization (SESI) mass spectrometry. SESI is an electrospray ionization technique carried out at atmospheric pressure. The term “SESI” generally covers a range of modified ESI techniques where the electrospray ionization plume ionises substances in the immediate vicinity of the electrospray plume. Typically, a SESI technique is carried out within a ionisation chamber in front of the skimmer entrance of an atmospheric pressure ionization mass spectrometer.

In one embodiment, the mass spectrometry is secondary ion mass spectrometry (SIMS). As is known to the person skilled in the art, SIMS is an MS method typically carried out on solid targets and thin films. The ionisation phase typically comprises sputtering the solid target surface with a primary ion beam, typically generated by a primary ion gun. A primary ion column may also be used to accelerate and focus the primary ion beam onto the target. When the ion beam strikes the target, this ejects secondary ions from the surface of the target. These secondary ions are then subjected to mass spectrometry using a mass analyser and a detector as described generally above. The mass analyser may be an electrostatic analyser, a quadrupole mass analyser, or a time of flight mass analyser. The detector may be a Faraday cup, an electron multiplier, or a microchannel plate detector.

In one embodiment, the mass spectrometry is carried out in full scan monitoring mode. As is known to the person skilled in the art, full scan monitoring involves scanning the mass range from the smallest the highest mass of ions expected (compared with selected ion monitoring mode in which data is only collected on the selected masses of interest).

In a preferred embodiment, the methods used in steps (c) and (d) are liquid chromatography coupled to tandem mass spectrometry. Using tandem mass spectrometry coupled to liquid chromatography, the analytes can be determined with high selectivity, flexibility and sensitivity.

In one particularly preferred embodiment, the chromatographic separation method used in step (c) is hydrophilic interaction liquid chromatography and the mass spectrometry method used in step (d) is triple quadrupole mass spectrometry. As described above, the combination of HILIC and triple quadrupole mass spectroscopy (especially with the mass spectrometer operating in MRM mode) enables a very selective, sensitive, and accurate measurement of nucleotides in complex matrices.

### Calculation of Capping Efficiency

Based on the concentrations of the analytes as measured using step (d), the RNA capping efficiency can be calculated.

Generally, the RNA capping efficiency can be measured according to the equation: [concentration of dinucleotide]/[concentration of dinucleotide+concentration of nucleotide].

When the uncapped hydrolysis product is GTP, the RNA capping efficiency is measured according to the equation: [concentration of dinucleotide]/[concentration of dinucleotide+ concentration of GTP].

When the uncapped hydrolysis product comprises both GTP and ATP, the RNA capping efficiency is measured according to the equation: [concentration of dinucleotide]/[concentration of dinucleotide+ concentration of GTP+ concentration of ATP].

### EXAMPLES

### Example 1: Enzymatic Hydrolysis

In this experiment RNA was enzymatically hydrolyzed to obtain single nucleotides (mononucleotide monophosphates and 5′ end mononucleotide triphosphate or 5′ dinucleotide of the 5′ cap). The obtained nucleotides were used for further analysis using LC-MS/MS (see Example 2).

### Enzymatic Hydrolysis of RNA

RNA was completely hydrolyzed by the action of one hydrolase. Nuclease P1 (NP1) from Penicillium citrinum was obtained as dried powder from Sigma Aldrich. NP1 was dissolved in 1 ml water to a concentration of about 1 mg/ml and stored at −20° C.

The RNA was filtered using Amicon Ultra 0.5 ml MWCO 30 kDa filters. Depending on the length of the RNA, 200-400 μg of RNA were hydrolyzed by adding 15 μl NH₄OAc buffer (100 mM pH 4.5) and 15 μl NP1 solution and incubating 3 h on a thermomixer at 37° C. and 450 rpm. 1 μl of the resulting solution was analyzed by LC-MS/MS (see Example 2).

### Example 2: LC-MS/MS Analysis of the Hydrolyzed RNA

In this experiment, the nucleotides obtained via enzymatic RNA hydrolysis (see Example 1) were analyzed by LC-MS/MS to determine the capping efficiency. This inventive method can be used as a quality control of in vitro transcribed RNA.

### 1. HPLC Analysis of Nucleotides

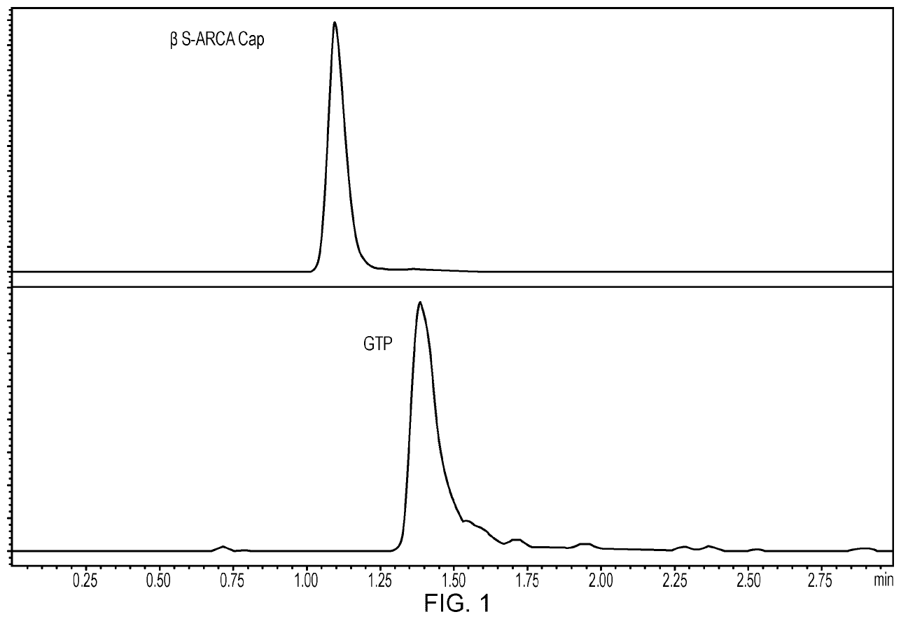

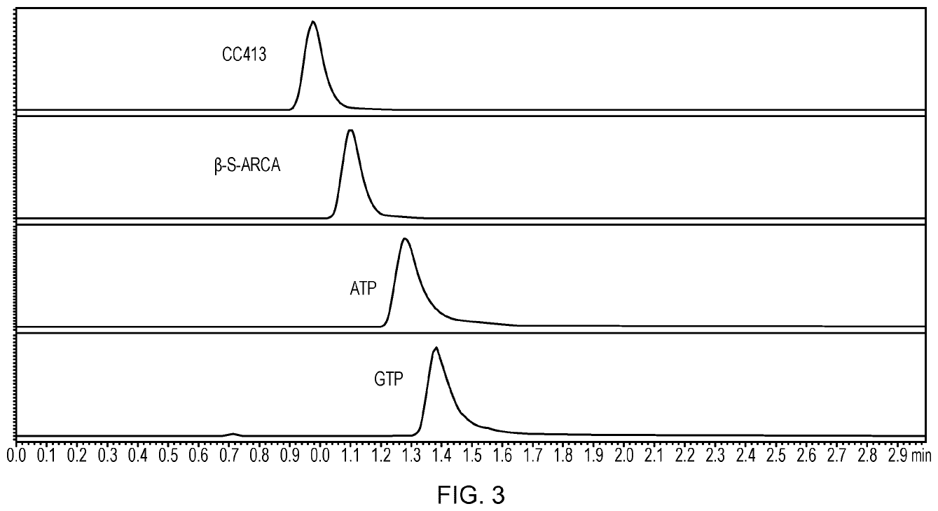

After enzymatic hydrolysis (see Example 1) RNA samples were separated using a commercially available HPLC setup. For the chromatographic separation of the nucleotides an amide column (Waters XBridge Premier BEH Amide VanGuard FIT Column, pore size: 130 Å, particle size: 2.5 μm, dimensions (h×ID) 2.1 mm×50 mm, Waters) and a linear HILIC gradient from 75% Buffer B (acetonitrile+0.1% water) to 55% Buffer A (ammonium carbonate 100 mM, pH 8.9) at 55° C. column temperature was used. For calibration, commercially available or commissioned standards were obtained. The nucleotides were detected by mass spectrometry. Exemplary chromatograms for a hydrolyzed β-S-ARCA-capped RNA, a hydrolyzed CleanCap® 413 capped RNA and respective standards are shown in FIG. 1, FIG. 2 and FIG. 3.

### 2. Mass Spectrometry of Nucleotides

The detection of the chromatographic peaks was performed by a commercially available (Shimadzu 8050) triple quadrupole mass spectrometer operated in multiple reaction monitoring (MRM) mode. Table 1 shows the mass transitions of nucleotides and caps used for quantification.

TABLE 1
Mass transitions

| Mass | Dwell | Q1 Pre | CE | Q3 Pre | Analyte |
|---|---|---|---|---|---|
| transition (u) | (ms) | Bias (V) | (V) | Bias | (V) |
| 523.80 > | 50.00 | −26.0 | −25.0 | −29.0 | GTP |
| 152.05 | | | | | |
| 833.10 > | 50.00 | −24.0 | −42.0 | −30.0 | β-S-ARCA |
| 166.20 | | | | | |
| 815.10 > | 50.00 | −22.0 | −55.0 | −30.0 | 7-Me-3′-OMe-Gppp- |
| 166.15 | | | | | 2′-OMe-A |
| 507.80 > | 50.00 | −24.0 | −55.0 | −27.0 | ATP |
| 136.10 | | | | | |

### 3. Quantification of the Nucleotide Concentrations

The nucleotide concentrations were calculated using isotopically labeled internal standards. The mononucleotide standards are commercially available; the dinucleotide standards were synthesized by a contract manufacturing organization (Hongene Biotech).

The area ratio of analyte's area to internal standard's area is used for calculation of calibration curves applying a linear or quadratic regression curve with appropriate weighting to the areas of the calibration samples by least squares analysis with the quantitation software of the LC-MS/MS/MS system.

### 4. Calculation of Capping Efficiency

The amount of the dinucleotide from the capping structure in the RNA hydrolysate was determined (Conc. (Cap)) as well as the amount of GTP (Conc. (GTP)) and ATP (Conc. (ATP)) which represent uncapped RNA. The percentage of capped RNA (Capping Efficiency %) can be determined in the respective samples, using one of the following formulas depending on the incorporated 5′ Cap:

$$Capping Efficiency ( \% ) = \frac{C o n c . ( Cap )}{Sum ( Conc . ( Cap ) + C o n c . ( GTP ) )} * 100$$

$$or$$

$$Capping Efficiency ( \% ) = \frac{C o n c . ( Cap )}{Sum ( Conc . ( Cap ) + C o n c . ( GTP ) + Conc . ( ATP ) )} * 100$$

The 5′ cap of RNA is an essential structure of protein coding RNAs, because non-capped RNA is not translated into protein. Therefore, determining the capping efficiency of in vitro transcribed RNA is a key quality control of in vitro transcribed RNA.

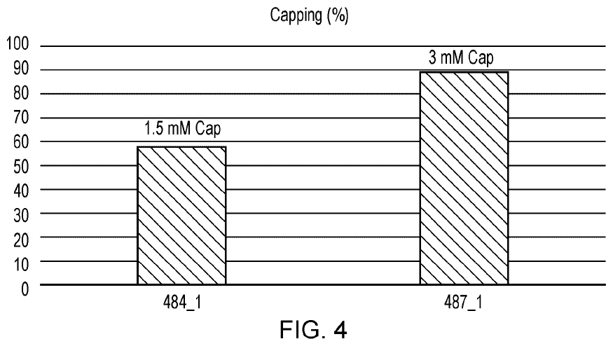

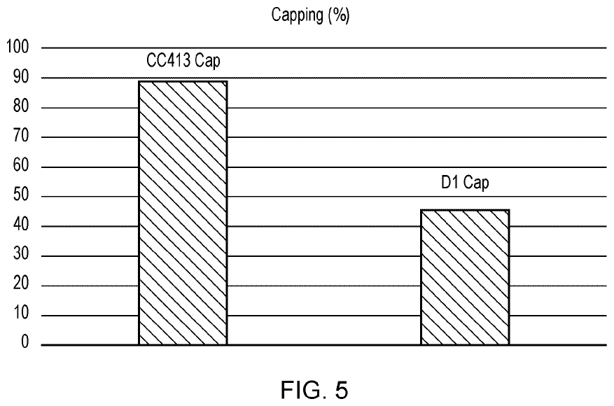

FIG. 4 illustrates the capping efficiency of varying CleanCap® 413 amounts in in vitro transcription, and FIG. 5 shows the capping efficiency of RNAs having two different cap structures, namely β-S-ARCA and CleanCap® 413. FIG. 4 and FIG. 5 illustrate that the inventive method is particularly suitable for measuring RNA quality attributes such as RNA capping efficiency for different amount of Cap in in vitro transcription (FIG. 4) and capping structures (FIG. 5) which showed different levels of capping efficiency. Capping is a key feature of RNA because non-capped RNA is not translated into proteins.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry, biochemistry, molecular biology, biotechnology or related fields are intended to be within the scope of the following claims.

1. A method of quantifying RNA capping efficiency, the method comprising the following steps (a) to (d):
(a) providing a sample of capped RNA;
(b) contacting the capped RNA with a nuclease, wherein the nuclease is a protein, thereby hydrolysing the RNA to produce hydrolysis products comprising a capped product comprising dinucleotides, and an uncapped product comprising nucleotides, step (b) being carried out in the absence of a nucleic acid having a base sequence complementary to the sequence of the capped RNA;
(c) separating the hydrolysis products by chromatography; and
(d) determining the concentrations of the hydrolysis products by triple quadrupole mass spectrometry, thereby quantifying RNA capping efficiency.

2. A method according to claim 1, wherein in step (b) the nuclease is the sole enzyme which hydrolyses the RNA.

3. A method according to claim 1 or claim 2, wherein the nuclease used in step (b) is nuclease P1 or nuclease S1.

4. A method according to claim 3, wherein the nuclease used in step (b) is nuclease P1.

5. A method according to any preceding claim, wherein the chromatography used in step (c) is liquid chromatography.

6. A method according to claim 5, wherein the chromatography used in step (c) is selected from the group consisting of high-performance liquid chromatography and ultra-performance liquid chromatography.

7. A method according to claim 5, wherein the chromatography used in step (c) is selected from the group consisting of hydrophilic interaction chromatography and ion-exchange chromatography.

8. A method according to claim 5, wherein the chromatography used in step (c) is hydrophilic interaction liquid chromatography.

9. A method according to claim 1, wherein the triple quadrupole mass spectrometry used in step (d) uses a multiple reaction monitoring (MRM) method.

10. A method according to any preceding claim, wherein the methods used in steps (c) and (d) are liquid chromatography coupled to triple quadrupole mass spectrometry.

11. A method according to claim 10, wherein the methods used in steps (c) and (d) are hydrophilic interaction liquid chromatography coupled to triple quadrupole mass spectrometry.

12. A method according to any preceding claim, wherein the RNA is mRNA.

13. A method according to any preceding claim, wherein the capped hydrolysis product is a dinucleotide.

14. The method of claim 13, wherein the capped hydrolysis product consists of a dinucleotide.

15. The method of claim 1, 13 or 14, wherein the dinucleotide comprises two nucleosides connected by a 5′,5′-triphosphate bridge.

16. The method of claim 1, wherein the capped hydrolysis product has a structure of formula (II): 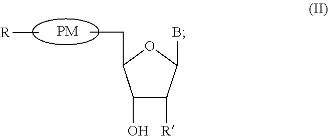
wherein:
PM is a mono- or polyphosphate moiety containing 1, 2, 3, 4, 5, 6, 7, 8, 9 or 10 phosphate units, the oxo (═O) groups in any of the phosphate units being optionally replaced with a thio (═S) group;
R is an end-cap moiety;
B is a nucleobase, optionally alkylated on a nitrogen atom by a C₁₋₄ alkyl group; and
R′ is selected from OH, O(C₁₋₄ alkyl), and halogen.

17. The method of claim 1, wherein the capped hydrolysis product has a structure of formula (IIa): 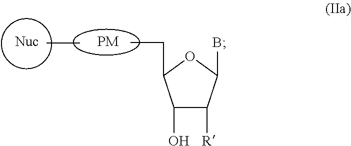
wherein:
PM is a mono- or polyphosphate moiety containing 1, 2, 3, 4, 5, 6, 7, 8, 9 or 10 phosphate units, the oxo (═O) groups in any of the phosphate units being optionally replaced with a thio (═S) group;
Nuc is a nucleoside or nucleoside analogue;
B is a nucleobase, optionally alkylated on a nitrogen atom by a C₁₋₄ alkyl group; and
R′ is selected from OH, O(C₁₋₄ alkyl), and halogen.

18. The method of claim 16, wherein the capped hydrolysis product has a structure of formula (IIb): 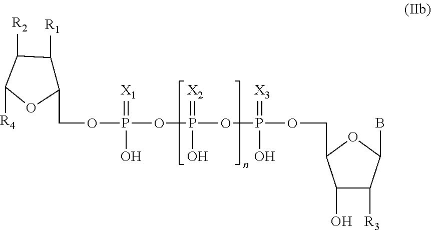
or a salt thereof, wherein:
B is a nucleobase, optionally alkylated on a nitrogen atom by a C₁₋₄ alkyl group;
R₁ is selected from OH, O(C₁₋₄ alkyl), and halogen;
R₂ is selected from H, OH, and O(C₁₋₄ alkyl), and halogen;
R₃ is selected from OH, O(C₁₋₄ alkyl), and halogen;
R₄ is H, OH, O(C₁₋₄ alkyl), halogen, or a nucleobase, optionally alkylated on a nitrogen atom by a C₁₋₄ alkyl group;
n is 1, 2 or 3; and
X₁, each X₂, and X₃, are each independently O or S.

19. The method of claim 18, wherein the capped hydrolysis product has a structure of formula (IIc): 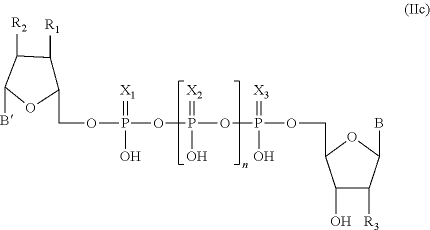
or a salt thereof, wherein:
B and B′ are each independently nucleobases, each optionally alkylated on a nitrogen atom by
a C₁₋₄ alkyl group;
R₁ is selected from OH, O(C₁₋₄ alkyl), and halogen;
R₂ is selected from H, OH, and O(C₁₋₄ alkyl), and halogen;
R₃ is selected from OH, O(C₁₋₄ alkyl), and halogen;
n is 1, 2 or 3; and
each X and X′ is independently O or S.

20. A method according to any preceding claim, wherein the capped hydrolysis products are selected from the group consisting of m7G(5′)ppp(5′)N, m7G(5′)ppp(5′)-(cap 0), m7G(5′)ppp(5′)Nm-(cap 1), m7G(5′)ppp(5′)G, ARCA, β-S-ARCA, wherein G is guanosine, p is a phosphate residue, N is any nucleoside, and Nm is a nucleoside having a 2′-methyl group.

21. The method of any preceding claim, wherein the dinucleotides comprise more than 50% by weight, such as more than 60% by weight, such as more than 70% by weight, such as more than 80% by weight, such as more than 90% by weight, such as more than 95% by weight, such as more than 96% by weight, such as more than 97% by weight, such as more than 98% by weight, such as more than 99% by weight, such as more than 99.5% by weight, such as more than 99.7% by weight, such as more than 99.9% by weight, such as more than 99.99% by weight, of the total weight of the capped hydrolysis products.

22. The method of any preceding claim, wherein nucleotides comprise more than 50% by weight, such as more than 60% by weight, such as more than 70% by weight, such as more than 80% by weight, such as more than 90% by weight, such as more than 95% by weight, such as more than 96% by weight, such as more than 97% by weight, such as more than 98% by weight, such as more than 99% by weight, such as more than 99.5% by weight, such as more than 99.7% by weight, such as more than 99.9% by weight, such as more than 99.99% by weight, of the total weight of the uncapped hydrolysis products.

23. A method according to claim 1, wherein the uncapped hydrolysis products include guanosine triphosphate (GTP) and the method includes determining the amount of GTP.

24. A method according to claim 23, wherein the RNA capping efficiency is measured according to the equation: [concentration of dinucleotide]/[concentration of dinucleotide+ concentration of GTP].

25. A method according to claim 1, wherein the uncapped hydrolysis products include adenosine triphosphate (ATP) and the method includes determining the amount of ATP.

26. A method according to claim 25, wherein the RNA capping efficiency is measured according to the equation: [concentration of dinucleotide]/[concentration of dinucleotide+concentration of GTP+concentration of ATP].

27. A method according to any preceding claim, wherein an external standard is used to determine the amount of the hydrolysis products.

28. A method according to any preceding claim, the method being carried out in the absence of a snake venom phosphodiesterase.

29. A method according to any preceding claim, the method being carried out in the absence of a phosphatase.

30. A method according to any preceding claim, wherein the capped RNA is purified prior to step (b).

31. A method according to claim 30, wherein the capped RNA is purified by filtration prior to step (b).

32. A method according to claim 30 or claim 31, wherein the capped RNA is purified by high performance liquid chromatography prior to step (b).

33. A method according to any preceding claim, wherein step (b) is carried out at a nuclease concentration of 10 to 70 μmol/L.

34. A method according to any preceding claim, wherein step (b) is carried out at a temperature of room temperature to 90° C.

35. A method according to any preceding claim, wherein step (b) is carried out for a time of 30 minutes to 48 hours.

36. A method according to any preceding claim, wherein step (b) is carried out at a pH of 4 to 6.

37. A method according to any one of claims 4 to 36, wherein in step (c) the mobile phase used in the liquid chromatography is a mixture of water and acetonitrile.

38. A method according to any one of claims 4 to 36, wherein the mobile phase used in the liquid chromatography is hexafluoroisopropanol.

39. A method according to any of claims 4 to 38, wherein the mobile phase used in the liquid chromatography includes a buffer.

40. A method according to claim 39, when the buffer is ammonium carbonate.

41. A method according to any of claims 4 to 39, wherein step (c) is carried out at a pH of 2 to 11.

42. A method according to claim 41, wherein step (c) is carried out at a pH of 8 to 10.

43. A method according to any one of claims 4 to 39, wherein step (c) is carried out at a flow rate of 0.1 to 2 ml/min.

44. A method according to claim 43, wherein step (c) is carried out at a flow rate of 0.3 to 1.0 ml/min.
